(19) United States
(12) Patent Application Publication
(10) Pub. No.: US 2025/0318546 A1
(43) Pub. Date: Oct. 16, 2025

(54) WATERCRESS EXTRACTION METHOD

(71) Applicant: University of Exeter, Exeter (GB)

(72) Inventors: Kyle James Stewart, Newton Abbot (GB); Paul Graham Winyard, Exeter (GB)

(21) Appl. No.: 18/257,090

(22) PCT Filed: Dec. 17, 2021
(86) PCT No.: PCT/EP2021/086662

§ 371 Date: Jun. 12, 2023

(30) Foreign Application Priority Data
Dec. 18, 2020 (GB) 2020180.2
Jul. 9, 2021 (GB) 2109921.3

(51) Int. Cl. A23J 3/14; A23B 2/90; A23B 7/06; A23J 1/00; A23L 33/185

(52) U.S. Cl. CPC A23J 3/14; A23B 2/90; A23B 7/06; A23J 1/007; A23L 33/185

(57) ABSTRACT

A method for preparing at least one watercress extract, which method comprises: (a) macerating watercress to form an aqueous watercress intermediate; (b) heating at least part of the aqueous watercress intermediate to coagulate at least a portion of the proteins in the watercress, forming a coagulated protein fraction and a liquid fraction; and (c) isolating the liquid fraction to provide an aqueous watercress extract and/or isolating the coagulated protein fraction to provide a watercress protein extract.

### TECHNICAL FIELD

This invention relates to methods for preparing at least one watercress extract. Aspects of the invention also relate to an aqueous watercress extract obtainable by the methods, and products obtainable by processing the extract, such as a fermented product obtainable by fermenting the aqueous watercress extract, a dried product obtainable by drying the aqueous watercress extract, and a downstream product comprising the aqueous watercress extract, the fermented product or the dried product; further aspects of the invention relate to these extracts and products for use in therapy, as well as uses in treatment for cosmetic purposes, in agricultural applications, as a food preservative and in odour control. Further aspects of the invention relate to a watercress protein extract obtainable by the methods, and products obtainable by processing the extract, such as a fermented product obtainable by fermenting the watercress protein extract, a dried product obtainable by drying the watercress protein extract, and a downstream product comprising the watercress protein extract, the fermented product or the dried product; further aspects of the invention relate to the use of these extracts and products in a food or drink product and as a food preservative.

### BACKGROUND

Watercress (Nasturtium officinale, also known as Rorippa nasturtium aquaticum) is an aquatic perennial plant that belongs to Brassicaceae family. It is a leaf vegetable commonly consumed by humans.

Watercress is known to contain a number of bioactive phytochemicals which can have benefits to humans. Such benefits include, for example, the potential antioxidant activity of carotenoids, chlorophyll and other phenolic compounds. Watercress is also known to have urease inhibitory activity.

Urease is an enzyme responsible for the rapid hydrolysis of urea to ammonia, which can be indicated by the following scheme:

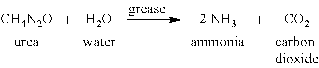

Urease is not produced by human or mammalian cells, but is present in bacteria, fungi and plants. In humans, urease produced by pathogenic and commensal (e.g. gut) bacteria can play a negative role in relation to a number of diseases and medical conditions. In the context of dermatitis (nappy rash), urease-producing bacteria present in the urine or on the skin convert urea to ammonia, and ammonia contributes to skin damage. The pathogenic bacterium, Helicobacter pylori (H. pylori) is associated with human gastric ulcers, gastric cancers, bleeds and lymphoma. H. pylori is able to survive in the harsh acidic conditions of the human stomach by urease-catalysed ammonia production, which creates a protective barrier in which the pH is neutralised. Struvite kidney stones are associated with infections with urease-positive organisms in the upper urinary tract. In liver cirrhosis, the failing liver is unable to perform fully its detoxification role of removing ammonia and other toxins generated by gut bacteria. Blood ammonia levels rise, cross the blood-brain barrier, and contribute to hepatic encephalopathy. The efficiency of the human gut absorption of nitrogen-which is essential for the synthesis of amino acids—is also influenced by urease: gut bacteria metabolise urea to ammonia, whilst human metabolism beneficially recycles urea into amino acids. Such metabolic pathways may be involved in age-related muscle loss (“sarcopenia”), which is a key contributor to frailty in the elderly population.

Through similar mechanisms in farm livestock, urease may play a role in lowering livestock yields. Urease may also contribute to diseases in domesticated animals, including livestock and domestic pets, through a number of the pathways described above for humans. Inhibition of urease activity is also of importance in agriculture. Inhibition of urease in livestock can play a role in increasing yield by improving gut absorption of amino acids. In the soil, inhibition of urease can improve the growth of some plants, while simultaneously reducing the environmental impact of ammonia produced through the use of some fertilisers.

The synthetic urease inhibitor, Lithostat® (acetohydroxamic acid) is used to treat recurrent urinary stones and recurrent urinary tract infections in the USA, but is associated with adverse side-effects. N-(n-butyl)thiophosphoric triamide (present in Agrotain® Plus Nitrogen Stabilizer) prevents ammonia volatilisation from soil via conversion to its oxon derivative which is a urease inhibitor.

A need remains for the development of easy and efficient processes for the recovery of urease inhibitors from plants such as watercress.

### SUMMARY OF THE INVENTION

An aspect of the invention provides a method for preparing at least one watercress extract, which method comprises:
(a) macerating watercress to form an aqueous watercress intermediate;
(b) heating at least part of the aqueous watercress intermediate to coagulate at least a portion of the proteins in the watercress, forming a coagulated protein fraction and a liquid fraction; and
(c) isolating the liquid fraction to provide an aqueous watercress extract and/or isolating the coagulated protein fraction to provide a watercress protein extract.

A further aspect of the invention provides an aqueous watercress extract obtainable by the methods of the invention.

Further aspects of the invention provide products obtainable by processing the aqueous watercress extract, for example by fermenting and/or drying the extract.

An aspect of the invention provides a fermented aqueous watercress product obtainable by fermenting the aqueous watercress extract of the invention.

An aspect of the invention provides a dried urease inhibitor (UI) product obtainable by drying, optionally freeze drying, the aqueous watercress extract of the invention or the fermented aqueous watercress product of the invention.

A further aspect of the invention provides a downstream product comprising the aqueous watercress extract of the invention, the fermented aqueous watercress product of the invention or the dried UI product of the invention.

Another aspect of the invention provides the aqueous watercress extract of the invention, the fermented aqueous watercress product of the invention or the dried UI product of the invention, for use in therapy.

A further aspect of the invention provides the use of the aqueous watercress extract of the invention, the fermented aqueous watercress product of the invention or the dried UI product of the invention, in treatment for cosmetic purposes.

Another aspect of the invention provides the use of the aqueous watercress extract of the invention, the fermented aqueous watercress product of the invention or the dried UI product of the invention, in agricultural applications.

A further aspect of the invention provides the use of the aqueous watercress extract of the invention, the fermented aqueous watercress product of the invention or the dried UI product of the invention, as a food preservative.

Another aspect of the invention provides the use of the aqueous watercress extract of the invention, the fermented aqueous watercress product of the invention or the dried UI product of the invention, in odour control.

A further aspect of the invention provides a watercress protein extract obtainable by the methods of the invention.

Further aspects of the invention provide products obtainable by processing the watercress protein extract, for example by fermenting and/or drying the extract.

An aspect of the invention provides a fermented protein product obtainable by fermenting the watercress protein extract of the invention.

An aspect of the invention provides a dried protein product obtainable by drying, optionally freeze drying, the watercress protein extract of the invention or the fermented protein product of the invention.

A further aspect of the invention provides a downstream product comprising the watercress protein extract of the invention, the fermented protein product of the invention or the dried protein product of the invention.

Another aspect of the invention provides the use of the watercress protein extract of the invention, the fermented protein product of the invention or the dried protein product of the invention, in a food product.

A further aspect of the invention provides the use of the watercress protein extract of the invention, the fermented protein product of the invention or the dried protein product of the invention, as a food preservative.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows a Roboqbo Qbo15-4 sample bowl equipped with cutting blade (left); loaded with 500 g of watercress (middle); loaded with 1 kg of watercress and chopped for 5 minutes at 2000 rpm (right). This figure relates to the blending apparatus used in Example 2.

FIG. 2 shows an extracted ion chromatogram (EIC), positive mode, (m/z=178.29)—this molecular ion is tentatively assigned to sulforaphane. This EIC was obtained from an analysis of the aqueous watercress extract by UHPLC-QTOF (as described in Example 2).

FIG. 3 shows an extracted ion chromatogram (EIC), negative mode (m/z=162.24)—this molecular ion is tentatively assigned to phenethylisothiocyanate (PEITC). This EIC was obtained from an analysis of the aqueous watercress extract by UHPLC-QTOF (as described in Example 2).

FIG. 4 shows A₆₇₀ of ammonium chloride calibration curve (y=0.0021x+0.3111, R²=1), as constructed for the urease inhibition assay (described in Example 2).

FIG. 5 shows cell viability calculated as % of MTT reduction to formazan compared to negative control treated with dPBS (described in Example 4). Panel A shows the results with all data points included. Panel B shows the results when outliers were removed from the 5% SDS and Extract E (referred to as “Extract IV” in Example 4) groups. In this figure, “dPBS” indicates the Dulbecco's phosphate-buffered saline group. “SDS” indicates the sodium dodecyl sulfate (also known as sodium lauryl sulfate) group.

FIG. 6 shows the amount of reduced formazan measured as absorbance at 540 nm (mean±SD) (described in Example 4). In this figure, “dPBS” indicates the Dulbecco's phosphate-buffered saline group. “SDS” indicates the sodium dodecyl sulfate (also known as sodium lauryl sulfate) group.

FIG. 7 shows the quantification of the release of the proinflammatory cytokine IL-1a after treatment (mean±SD) (described in Example 4). The enzyme-linked immunosorbent assay (ELISA) kit minimum detectable concentration of human IL-1α was typically less than 1.0 pg·mL⁻¹. In this figure, “dPBS” indicates the Dulbecco's phosphate-buffered saline group. “SDS” indicates the sodium dodecyl sulfate (also known as sodium lauryl sulfate) group.

FIG. 8 shows the time-course of the average skin irritancy score in volunteers, comparing the aqueous watercress extract (mixed 1:1 v/v with 0.3% SLS) with the positive control (0.3% SLS mixed 1:1 with distilled water) (described in Example 5). In this figure, the curve labelled “101” represents the positive control group (also labelled “Positive”). The curve labelled “102” represents the investigational sample group (also labelled “Invest”). Each data point represents the mean of the results obtained from n=30 volunteers.

FIG. 9 shows the time-course of the average skin irritancy score in volunteers, comparing a preparation of 10% v/v aqueous watercress extract in distilled water (mixed 1:1 v/v with 0.3% SLS) with the positive control (0.3% SLS mixed 1:1 with distilled water) (described in Example 5). In this figure, the curve labelled “103” represents the positive control group (also labelled “Positive”). The curve labelled “104” represents the investigational sample group (also labelled “Invest”). Each data point represents the mean of the results obtained from 30 volunteers.

FIG. 10 shows the time-course of the average skin irritancy score in volunteers, comparing a preparation of 10% v/v aqueous watercress extract in the carrier cream (mixed 1:1 v/v with 0.3% SLS) with the positive control (0.3% SLS mixed 1:1 with distilled water) (described in Example 5). In this figure, the curve labelled “105” represents the positive control group (also labelled “Positive”). The curve labelled “106” represents the investigational sample group (also labelled “Invest”). Each data point represents the mean of the results obtained from 30 volunteers.

FIG. 11 shows the time-course of the average skin irritancy score in SLS-exposed volunteers, demonstrating the effect of an intervention (at 48 h, in one of the two groups) with aqueous watercress extract (mixed 1:1 v/v with distilled water) (described in Example 5). In this figure, the curve labelled “107” represents the positive control group (also labelled “Positive”). The curve labelled “108” represents the investigational sample group (also labelled “Invest”). In the positive control group, 0.3% SLS, mixed 1:1 v/v with distilled water, was applied to the skin and left for 48 h; following this, distilled water was applied to the same skin area and left for 48 h. In the investigational sample group 0.3% SLS, mixed 1:1 v/v with distilled water, was applied to the skin and left for 48 h; following this, the aqueous watercress extract (neat) was applied to the same skin area and left for 48 h. Each data point represents the mean of the results obtained from 30 volunteers.

FIG. 12 shows the time-course of the average skin irritancy score in SLS-exposed volunteers, demonstrating the effect of an intervention (at 48 h, in one of the two groups) with 10% (v/v) aqueous watercress extract (mixed 1:1 v/v with distilled water) (described in Example 5). In this figure, the curve labelled “109” represents the positive control group (also labelled “Positive”). The curve labelled “110” represents the investigational sample group (also labelled “Invest”). In the positive control group, 0.3% SLS, mixed 1:1 v/v with distilled water, was applied to the skin and left for 48 h; following this, distilled water was applied to the same skin area and left for 48 h. In the investigational sample group 0.3% SLS, mixed 1:1 v/v with distilled water, was applied to the skin and left for 48 h; following this, 10% aqueous watercress extract, mixed 1:1 with distilled water, was applied to the same skin area and left for 48 h. Each data point represents the mean of the results obtained from 30 volunteers.

FIG. 13 shows the time-course of the average skin irritancy score in SLS-exposed volunteers, demonstrating the effect of an intervention (at 48 h, in one of the two groups) with 10% (v/v) aqueous watercress extract in a carrier cream (described in Example 5). In this figure, the curve labelled “111” represents the positive control group (also labelled “Positive”). The curve labelled “112” represents the investigational sample group (also labelled “Invest”). In the positive control group, 0.3% SLS, mixed 1:1 v/v with distilled water, was applied to the skin and left for 48 h; following this, distilled water was applied to the same skin area and left for 48 h. In the investigational sample group 0.3% SLS, mixed 1:1 v/v with distilled water, was applied to the skin and left for 48 h; following this, 10% aqueous watercress extract in a carrier cream, mixed 1:1 with distilled water, was applied to the same skin area and left for 48 h. Each data point represents the mean of the results obtained from 30 volunteers.

FIG. 14 shows the concentration-response curve when increasing concentrations of the aqueous watercress extract (expressed as a percentage of the neat aqueous watercress extract) were added to a solution of ammonium chloride at pH 7.4.

FIG. 15 shows 100 g of oven dried powdered whole watercress leaves and stems bought from The Watercress Company in Dorchester, UK (www.thewatercresscompany.com).

FIG. 16 shows the pan of dried watercress powder in FIG. 15 mixed with 3 litres of water to form a granular green mixture with powder particles clearly seen separate to the water.

FIG. 17 shows the result of heating the mixture in FIG. 16 for 2 hours at 80° C. in a pan, by which time the mixture had darkened.

FIG. 18 shows the disintegrated solid matter which was filtered out when the mixture in FIG. 17 was poured through a clean coffee filter.

FIG. 19 shows the brown liquid which passed through when the mixture described in FIG. 17 was poured through a clean coffee filter.

FIG. 20 shows 3 and ⅝ ounces of fresh watercress plant leaves and stems when put in a pan with one pint of water.

FIG. 21 shows the intact green stems and leaves recovered when the mixture described in FIG. 20 was heated to boiling point, allowed to simmer for 5 minutes, cooled and then strained through a fine muslin cloth. These were collected in the cloth.

FIG. 22 shows the brown transparent liquid which was collected when the mixture described in FIG. 20 was heated to boiling point, allowed to simmer for 5 minutes, cooled and then strained through a fine muslin cloth. The liquid passed through the cloth and was collected into a vessel.

FIG. 23 shows the thick green homogeneous mixture formed from the rough chopping and subsequent blending for 3 minutes using a Kenwood FP 195 food processor of fresh watercress plants (stems and leaves).

FIG. 24 shows the insoluble fibre when the liquid in FIG. 23 was passed through a muslin cloth.

FIG. 25 shows a close-up of the insoluble fibre in FIG. 24.

FIG. 26 shows a bright green liquid formed when the thick green mixture in FIG. 23 was passed through a muslin cloth to remove the fibre.

FIG. 27 shows the coagulation effect when the liquid in FIG. 26 was heated in a pan to approximately 80° C.

FIG. 28 shows a spoonful of the pan contents in FIG. 27 demonstrating a green putty-like solid component separate from a transparent brown liquid.

FIG. 29 shows a close-up of the putty-like green coagulant recovered when the heated mixture in FIG. 27 was poured through a cloth. This putty was recovered from the cloth.

FIG. 30 shows a brown liquid which has passed through a cloth when the heated mixture in FIG. 27 was poured through it.

FIG. 31 shows 300 ml of water blended for 10 minutes with 300 g oven dried watercress bought from The Watercress Company (www.the watercress company.com) using a Kenwood FP 195 food processor, being passed through a coffee filter to collect a brown transparent liquid.

FIG. 32 shows the third coffee filter which the brown transparent liquid shown in FIG. 31 has been passed through demonstrating there is no particulate matter filtered out.

FIG. 33 shows the brown transparent liquid described in FIG. 31 at 80° C. There is no evidence of a coagulation effect or separation/creation of a solid component.

FIG. 34 shows the coffee filter after the heated liquid in FIG. 33 was poured through. There is no solids retained showing that no solids formed during the heating process.

### DETAILED DESCRIPTION

Aspects of the invention provide methods for preparing at least one watercress extract, which comprise the steps of macerating watercress, heating the product, and isolating at least one resulting extract.

In an embodiment, the invention provides a method for preparing at least one watercress extract, which method comprises:
(a) macerating watercress to form an aqueous watercress intermediate;
(b) heating at least part of the aqueous watercress intermediate to coagulate at least a portion of the proteins in the watercress, forming a coagulated protein fraction and a liquid fraction; and
(c) isolating the liquid fraction to provide an aqueous watercress extract and/or isolating the coagulated protein fraction to provide a watercress protein extract.

The inventors have found that the heating step in this method surprisingly results in the coagulation of proteins present in the aqueous watercress intermediate formed by macerating watercress. Without wishing to be bound by theory, it is thought that the heat may be denaturing some of the proteins, causing them to coagulate. The coagulated proteins can precipitate as a solid or semi-solid material, forming at least part of the coagulated protein fraction, which can easily be separated from the liquid fraction.

In an embodiment, the liquid fraction may be isolated to provide the aqueous watercress extract.

In another embodiment, the coagulated protein fraction may be isolated to provide the watercress protein extract.

Preferably, the liquid fraction and the coagulated protein fraction are isolated by separating the coagulated protein fraction and the liquid fraction, to form both the watercress protein extract and the aqueous watercress extract. The separation of the coagulated protein fraction and the liquid fraction can for example be performed by filtration. The aqueous watercress extract may pass through the filter, forming the filtrate, while the watercress protein extract may be retained by the filter, forming the residue.

Suitably, the aqueous watercress extract comprises at least one urease inhibitor and/or has urease inhibitory activity. The aqueous watercress extract may also be referred to as a watercress liquid extract, or as a urease inhibiting liquid. The inventors have surprisingly found that a substantial proportion of all the urease inhibitory activity found in watercress is attributable to low molecular weight compounds which are hydrophilic and/or water soluble.

The method of the invention provides an efficient and convenient method for providing an extract comprising urease inhibitors from watercress, in a sustainable fashion.

In addition, the method of the invention can provide both the aqueous watercress extract and the watercress protein extract at the same time in one efficient method.

The methods of the invention comprise the step (a) of macerating watercress to form an aqueous watercress intermediate.

As used herein, the term “macerating” means any process by which the physical structure of the watercress is broken up.

Suitably, macerating the watercress may comprise macerating a watercress component comprising or consisting of the watercress.

The watercress may of course be macerated in the presence of suitable additives, such as for example a liquid maceration medium, which may e.g. comprise or consist of water.

In some embodiments, macerating the watercress may comprise macerating a watercress component comprising the watercress and a liquid maceration medium.

In a preferred embodiment, the maceration step may result in the rupture of plant cells, also referred to as cellular disruption. Suitably, the maceration step may lead to an increased level of cellular disruption.

The macerating step may be conducted by any known method for macerating (breaking up) a plant material, which would be well-known to a person skilled in the art.

Suitably, the macerating step may comprise contacting the watercress with a blade, for example a spinning blade. The rotational speed of such a spinning blade may, for example, be in the range of from 500 rpm or from 1000; and/or up to 50000 rpm, up to 40000 rpm, or up to 30000 rpm. Preferably, the rotational speed may be in the range of from 1000 rpm to 40000 rpm, or from 1000 rpm to 30000 rpm.

In an embodiment, the macerating step may be conducted for a time period in the range from 30 seconds to 1 hour. Preferably, the macerating step is conducted for a time period in the range from 1 minute to 30 minutes, or from 2 minutes to 10 minutes. Most preferably, the macerating step is conducted for about 5 minutes.

Suitably, the maceration step may result in the aqueous watercress intermediate comprising watercress pieces with a largest diameter size of 5 mm or less, 4 mm or less, 3 mm or less or 2 mm or less, preferably 1 mm or less, and more preferably 0.9 mm or less, 0.8 mm or less, 0.7 mm or less, 0.6 mm or less, 0.5 mm or less, 0.4 mm or less, 0.3 mm or less, 0.2 mm or less, or 0.1 mm or less. These sizes can for example be determined by taking a sample and making a visual determination, optionally with a microscope.

The macerating step may for example be conducted in a commercial mixer, grinder or blender, such as a kitchen blender. Optionally, the maceration step may be conducted as a sequence of steps which achieve increasingly smaller sizes of pieces, for example as an initial stage of roughly chopping up the watercress, followed by a step in a course blender and a subsequent step in a finer blender. Such techniques would be well known to a person skilled in the art.

The aqueous watercress intermediate can also be referred to as a macerated watercress intermediate, an aqueous watercress paste, a macerated watercress paste, or a macerated watercress mixture.

The aqueous watercress intermediate contains water which was present in the original watercress, as well as other components which were present in the original watercress. Optionally, additional water may be added to the watercress intermediate to adjust the viscosity.

The aqueous watercress intermediate may be an aqueous suspension of macerated watercress tissue, which can be cell fragments and cell constituents in water.

In an embodiment, the step of macerating the watercress can comprise blending the watercress or be a step of blending the watercress. The resulting aqueous watercress intermediate can also be referred to as a watercress blend, and aqueous watercress blend, or blended watercress.

In an embodiment, the step of macerating the watercress can comprise homogenising the watercress or be a step of homogenising the watercress. In such an embodiment, the maceration step may result in homogenisation of the plant tissue (watercress tissue). The resulting aqueous watercress intermediate can also be referred to as a homogeneous mixture or homogenate.

Optionally, the method of the invention may comprise removing solids from the aqueous watercress intermediate before heating. This removal of solids may result in a watercress liquid, which watercress liquid can be heated to coagulate at least a portion of the proteins in watercress, forming the coagulated protein fraction and the liquid fraction.

By using the optional step of removing solids from the aqueous watercress intermediate before the heating step, some of the fibrous material which is present in the original watercress may be removed before the proteins are coagulated. This may result in a coagulated protein fraction containing less fibrous material, and therefore having a larger weight percentage of proteins. After the isolation step (c), this can result in a watercress protein extract containing less fibrous material, and therefore containing a larger weight percentage of proteins, than without the use of the optional step of removing solids from the aqueous watercress intermediate before heating. The watercress protein extract containing less fibrous material may be described as a purer protein extract.

For example, in an embodiment without the optional step of removing solids from the aqueous watercress intermediate before the heating step, the resulting watercress protein extract may comprise up to 80 wt % protein, up to 70 wt % protein, up to 60 wt % protein, up to 50 wt % protein, or up to 40 wt % protein, such as about 40 wt % protein. The wt % of protein may, for example, be measured by a biochemical protein assay such as the Kjeldahl method.

In an embodiment with the optional step of removing solids from the aqueous watercress intermediate before the heating step, the resulting watercress protein extract may comprise at least 30 wt % protein, at least 40 wt % protein, at least 50 wt % protein, at least 60 wt % protein, at least 70 wt % protein, or at least 80 wt % protein. The wt % of protein may, for example, be measured by a biochemical protein assay such as the Kjeldahl method.

The optional step of removing the solids, which solids can e.g. be watercress fibre or pulp, from the aqueous watercress intermediate before heating may be conducted by any known method for separating solids and liquids, which would be well-known to a person skilled in the art. For example, the solids may be removed by pressing the aqueous watercress intermediate and isolating the liquid (or juice), by filtering the aqueous intermediate and isolating the filtrate, or by centrifuging the aqueous intermediate and isolating the supernatant.

Filtration of the aqueous watercress intermediate to remove solids may for example be conducted by using any filtration device, such as a commercial filtration device, or by using a filter such as e.g. a muslin cloth, cheese cloth, sacking material, nylon mesh, wire mesh, or filter paper. The filtration may optionally be conducted under pressure. In an embodiment, the filtration of the aqueous watercress intermediate is conducted using a muslin filter, for example having a pore size of up to 2 mm. In another embodiment, the filtration may be conducted using a filter, e.g. a (commercial) membrane filter, with a pore size of up to 0.45 μm, up to 0.22 μm, or up to 0.2 μm; and/or from 0.1 μm. Preferably, the pore size of the filter may be in the range of from 0.1 μm to 0.45 μm, more preferably from 0.1 μm to 0.22 μm, or from 0.1 μm to 0.2 μm. The pore size of the filter may, for example, be around 0.2 μm.

In an embodiment, the filtration may be conducted by using a filtration through a filter with a larger pore size (such as a pore size of up to 2 mm, and/or from 0.5 μm), followed by a filtration through a filter with a smaller pore size (such as a pore size of up to 0.45 μm, up to 0.22 μm, or up to 0.2 μm, optionally from 0.1 μm).

Centrifugation of the aqueous watercress intermediate may for example be conducted by using a commercial centrifuge. Centrifugation may result in a pellet of solid in a centrifugal vessel with a supernatant above it. The solids which are being removed may be found in the pellet, while the supernatant may be isolated for subsequent processing.

The methods of the invention comprise the step (b) of heating at least part of the aqueous watercress intermediate to coagulate at least a portion of the proteins in watercress.

In an embodiment, the entire aqueous watercress intermediate is heated to coagulate at least a portion of the proteins in watercress, forming a coagulated protein fraction and a liquid fraction.

In another embodiment, part of the aqueous watercress intermediate is heated to coagulate at least a portion of the proteins in watercress, forming a coagulated protein fraction and a liquid fraction.

In a preferred embodiment, a liquid part of the aqueous watercress intermediate is heated, which liquid part may be the watercress liquid which can be obtained after removal of solids from the aqueous watercress intermediate as described above.

In an embodiment, a time period between the end of the maceration step (a) and the start of the heating step (b) may fall within a preferred range. This may allow desired chemical reactions to occur between the rupture of plant cells during the maceration step and the subsequent heating step, for example the formation of isothiocyanates such as sulforaphane and phenethyl isothiocyanate (PEITC). In an embodiment, the time period between the end of the maceration step (a) and the heating step (b) is at least 1 minute, at least 5 minutes, at least 10 minutes, at least 20 minutes, at least 30 minutes, at least 40 minutes, at least 50 minutes, at least 1 hour, at least 90 minutes, or at least 2 hours. Preferably, the time period is at least 10 minutes, at least 20 minutes, at least 30 minutes, at least 40 minutes, at least 50 minutes, at least 1 hour, at least 90 minutes, or at least 2 hours. More preferably, the time period is at least 30 minutes, at least 1 hour, at least 90 minutes, or at least 2 hours.

Preferably, the heating step is conducted at a temperature in the range from 50° C., from 60° C., or from 70° C.; and/or up to 200° C., up to 190° C., up to 180° C., up to 170° C., up to 160° C., up to 150° C., up to 140° C., up to 130° C., up to 120° C., up to 110° C., up to 100° C., up to 90° C., or up to 80° C. More preferably, the heating step is conducted at a temperature in the range from 50° C. to 200° C., or from 50° C. to 120° C., or from 60° C. to 90° C. Most preferably, the heating step is conducted at a temperature in the range from 70° C. to 80° C. Optionally, heating may be for any of the time periods indicated below.

Suitably, the heating step is conducted for a time period sufficient for the protein to coagulate.

In an embodiment, the heating step may be conducted for a time period in the range from 2 seconds to 2 hours. Suitably, this is the time period for which the material is held at an indicated stated temperature. Preferably, the heating step is conducted for a time period in the range from 10 seconds to 90 minutes, or from 20 seconds to 60 minutes, or from 30 seconds to 30 minutes, or from 1 minute to 5 minutes. Most preferably, the heating step is conducted for about 2 minutes.

The heating step may be conducted by any known method for heating an aqueous plant intermediate or an aqueous plant paste, which would be well-known to a person skilled in the art. For example, the aqueous watercress intermediate may be heated using a water bath, a pressure cooker, a steam cooker, an electro-thermal heating system, an electrically heated steel vessel, or thermal blanching.

After the heating step, the methods of the invention comprise the step (c) of isolating the liquid fraction to provide an aqueous watercress extract and/or isolating the coagulated protein fraction to provide a watercress protein extract.

The isolation step may be conducted by any known method for separating solids and liquids, which would be well-known to a person skilled in the art. One or both of the extracts may, for example, be isolated by filtration or by centrifugation.

In an embodiment, the liquid fraction may be isolated to provide the aqueous watercress extract. For example, the liquid fraction may be isolated by filtration or centrifugation to provide the aqueous watercress extract.

Suitably, the liquid fraction may be isolated by filtration to provide the aqueous watercress extract. The filtration may for example be conducted by using any filtration device, such as a commercial filtration device, or by using a filter such as e.g. a muslin cloth, cheese cloth, sacking material, nylon mesh, wire mesh, or filter paper. The filtration may optionally be conducted under pressure. Filtration can provide the aqueous watercress extract as the filtrate. In an embodiment, the filtration is conducted using a muslin filter, for example having a pore size of up to 2 mm. In another embodiment, the filtration may be conducted using a filter, e.g. a (commercial) membrane filter, with a pore size of up to 0.45 μm, up to 0.22 μm, or up to 0.2 μm; and/or from 0.1 μm. Preferably, the pore size of the filter may be in the range of from 0.1 μm to 0.45 μm, more preferably from 0.1 μm to 0.22 μm, or from 0.1 μm to 0.2 μm. The pore size of the filter may, for example, be around 0.2 μm.

Alternatively, the liquid fraction may be isolated by centrifugation to provide the aqueous watercress extract. The centrifugation may for example be conducted by using a commercial centrifuge. Centrifugation may result in a pellet of solid in a centrifugal vessel with a supernatant above it. Centrifugation can provide the aqueous watercress extract as the supernatant.

In another embodiment, the coagulated protein fraction may be isolated to provide the watercress protein extract. For example, the coagulated protein fraction may be isolated by filtration or centrifugation to provide the watercress protein extract.

Suitably, the coagulated protein fraction may be isolated by filtration to provide a watercress protein extract. The filtration may for example be conducted by using any filtration device, such as a commercial filtration device, or by using a filter such as e.g. a muslin cloth, cheese cloth, sacking material, nylon mesh, wire mesh, or filter paper. The filtration may optionally be conducted under pressure. Filtration can provide the watercress protein extract as the residue. In an embodiment, the filtration is conducted using a muslin filter, for example having a pore size of up to 2 mm. In another embodiment, the filtration may be conducted using a filter, e.g. a (commercial) membrane filter, with a pore size of up to 0.45 μm, up to 0.22 μm, or up to 0.2 μm; and/or from 0.1 μm. Preferably, the pore size of the filter may be in the range of from 0.1 μm to 0.45 μm, more preferably from 0.1 μm to 0.22 μm, or from 0.1 μm to 0.2 μm. The pore size of the filter may, for example, be around 0.2 μm.

Alternatively, the coagulated protein fraction may be isolated by centrifugation to provide the watercress protein extract. The centrifugation may for example be conducted by using a commercial centrifuge. Centrifugation may result in a pellet of solid in a centrifugal vessel with a supernatant above it. Centrifugation can provide the watercress protein extract as the solid pellet.

In a preferred embodiment, the liquid fraction may be isolated to provide the aqueous watercress extract and the coagulated protein fraction may be isolated to provide the watercress protein extract. The liquid fraction and the coagulated protein fraction may be isolated by separating the coagulated protein fraction and the liquid fraction, to form both the watercress protein extract and the aqueous watercress extract.

Suitably, the separation of the coagulated protein fraction and the liquid fraction may be conducted by filtration. The filtration may for example be conducted by using any filtration device, such as a commercial filtration device, or by using a filter such as e.g. a muslin cloth, cheese cloth, sacking material, nylon mesh, wire mesh, or filter paper. The filtration may optionally be conducted under pressure. Filtration can provide the aqueous watercress extract as the filtrate, and the watercress protein extract as the residue. In an embodiment, the filtration is conducted using a muslin filter, for example having a pore size of up to 2 mm. In another embodiment, the filtration may be conducted using a filter, e.g. a (commercial) membrane filter, with a pore size of up to 0.45 μm, up to 0.22 μm, or up to 0.2 μm; and/or from 0.1 μm. Preferably, the pore size of the filter may be in the range of from 0.1 μm to 0.45 μm, more preferably from 0.1 μm to 0.22 μm, or from 0.1 μm to 0.2 μm. The pore size of the filter may, for example, be around 0.2 μm.

Alternatively, the separation of the coagulated protein fraction and the liquid fraction may be conducted by centrifugation. The centrifugation may for example be conducted by using a commercial centrifuge. Centrifugation may result in a pellet of solid in a centrifugal vessel with a supernatant above it. Centrifugation can provide the aqueous watercress extract as the supernatant and the watercress protein extract as the solid pellet.

The methods of the invention may further comprise the optional step of sterilising one or more of the extracts.

In an embodiment, the methods may further comprise the step of sterilising the aqueous watercress extract and/or the watercress protein extract. This may be done to remove any microbes present.

The sterilising step may, for example, be conducted by filtration, centrifugation, autoclaving, gamma-irradiation, or any combination thereof. Preferably, the sterilising step may be conducted by filtration, autoclaving, gamma-irradiation, or any combination thereof, optionally in combination with centrifugation.

In a preferred embodiment, the aqueous watercress extract may be sterilised by filtration.

When filtration is used, in an embodiment the filtration may be conducted using a filter with a pore size of up to 0.45 μm, up to 0.22 μm, or up to 0.2 μm; and/or from 0.1 μm. Preferably, the pore size of the filter may be in the range of from 0.1 μm to 0.45 μm, more preferably from 0.1 μm to 0.22 μm, or from 0.1 μm to 0.2 μm. The pore size of the filter may, for example, be around 0.2 μm.

In a further embodiment, the watercress protein extract may be sterilised by gamma-irradiation.

In a further embodiment, the watercress protein extract may be sterilised by autoclaving.

After isolation step (c), and/or optionally after any sterilisation step, the methods of the invention may optionally comprise one or more further processing steps to prepare a downstream product from one or more of the watercress extracts. Further processing steps may comprise, for example, drying the extract, or fermenting the extract.

In an embodiment, the methods may further comprise the step of drying the aqueous watercress extract and/or the watercress protein extract. Preferably, the drying may be freeze drying.

The drying (preferably freeze drying) step may be conducted by any known method for drying, which would be well-known to a person skilled in the art.

In an embodiment, the aqueous watercress extract may be dried, preferably freeze-dried or spray-dried.

In another embodiment, the watercress protein extract may be dried, preferably freeze-dried or spray-dried.

Suitably, both the aqueous watercress extract and the watercress protein extract are dried, preferably freeze-dried.

In an embodiment, the methods of the invention may optionally comprise the step of fermenting the aqueous watercress extract and/or the watercress protein extract. Suitably, the fermenting may be conducted by incubating the extract with nitrate-reducing bacteria, nitrate-reductase enzymes and/or nitrate reducing chemicals. Without wishing to be bound by theory, fermentation of the extract may increase the nitrite content of the extract, which can be beneficial in meat preservation.

The fermenting step may be conducted by any known method for fermenting, which would be well-known to a person skilled in the art.

In an embodiment, the aqueous watercress extract may be fermented, for example by incubating the extract with nitrate-reducing bacteria, nitrate-reductase enzymes and/or nitrate reducing chemicals.

In another embodiment, the watercress protein extract may be fermented, for example by incubating the extract with nitrate-reducing bacteria, nitrate-reductase enzymes and/or nitrate reducing chemicals.

The fermentation step may be carried out before or after the optional sterilisation step, and before or after a drying step, for example by adding water to the dried product before fermentation.

The sterilisation step may be carried out before and/or after the optional fermentation step, and before and/or after the optional drying step.

In an embodiment, the fermentation step may be carried out before the optional drying step.

In an embodiment, the drying step may be carried out after the optional sterilisation step and/or after the optional fermentation step.

In an embodiment, the methods of the invention may optionally comprise the step of blanching the watercress prior to the maceration step (a). The blanching step may help to provide a greener product, which can be more acceptable to consumers.

In an embodiment, the methods of the invention may optionally comprise the step of adding chemical additives to the watercress intermediate prior to the heating step. Such additives may help to provide a greener product, which can be more acceptable to consumers. The chemical additives may, for example, be selected from water-soluble salts of bicarbonate (e.g. sodium bicarbonate and/or calcium bicarbonate), water-soluble metal chelating agents (e.g. the free acid of EDTA and/or a water-soluble salt of EDTA, such as disodium EDTA dihydrate, dipotassium EDTA dihydrate or tetrasodium EDTA tetrahydrate), and any combination thereof.

As aforesaid, watercress is a commonly known plant. The species of watercress used is Nasturtium officinale.

As aforesaid, macerating the watercress may comprise macerating a watercress component comprising or consisting of the watercress. Such a watercress component may comprise one or more suitable forms of watercress.

Suitably, the watercress which is used in the method of the invention is fresh watercress. The watercress may have been stored under conventional chilled conditions, or may have been frozen before use.

Watercress comprises watercress proteins. Suitably, the watercress may be “fresh” in the sense that watercress proteins are present in their natural form, i.e. are not denatured.

It has been found that proteins which are already in a denatured state cannot lead to the formation of a coagulated protein fraction in step (b).

Using dried watercress powder and water has been found not to result in coagulation on heating. Without wishing to be bound by theory, it is thought that in a dried watercress powder, the protein may already be in a denatured state and therefore is no longer soluble in water. The coagulation effect may also be dependent on mobilising the protein through macerating the watercress.

Macerating the watercress may comprise macerating a watercress component comprising or consisting of the watercress, wherein at least a majority, and optionally substantially the entirety, of watercress protein in the component is not denatured. Suitably, macerating the watercress may comprise macerating a watercress component that is substantially free from denatured watercress protein.

Another aspect of the invention provides an aqueous watercress extract obtainable by the method of the invention described above.

The aqueous watercress extract may comprise or consist of the liquid fraction.

Suitably, the aqueous watercress extract comprises at least one urease inhibitor (UI). Such urease inhibitors can, for example, comprise isothiocyanates, such as sulforaphane; patchouli alcohol; ascorbic acid; transition metal ions; and/or polyphenols. In an embodiment, the at least one urease inhibitor comprises isothiocyanates, for example sulforaphane; and/or patchouli alcohol. Preferably, the at least one urease inhibitor comprises isothiocyanates, for example sulforaphane.

In an embodiment, the aqueous watercress extract has urease inhibitory activity. This activity can, for example, be provided by the presence in the aqueous watercress extract of isothiocyanates, such as sulforaphane; patchouli alcohol; ascorbic acid; transition metal ions; and/or polyphenols. In an embodiment, the urease inhibitory activity is provided by isothiocyanates, for example sulforaphane; and/or patchouli alcohol. Preferably, the urease inhibitory activity is provided by isothiocyanates, for example sulforaphane.

In an embodiment, the aqueous watercress extract comprises (or further comprises) phenethyl isothiocyanate (PEITC).

Another aspect of the invention provides a fermented aqueous watercress product obtainable by fermenting the aqueous watercress extract of the invention.

A further aspect of the invention provides a dried urease inhibitor (UI) product obtainable by drying, preferably freeze drying, the aqueous watercress extract of the invention or the fermented aqueous watercress product of the invention.

The watercress extracts of the invention and the products obtainable by further processing of those extracts have a variety of uses, for example in therapy, cosmetic treatments, and food and drink technologies such as preservation particularly of meat.

An aspect of the invention provides a downstream product, such as a pharmaceutical or cosmetic composition, a food or drink product, comprising an extract of the invention, or a product obtainable by further processing of that extract.

Another aspect of the invention provides a downstream product comprising the aqueous watercress extract of the invention, the fermented aqueous watercress product of the invention, the dried UI product of the invention or a product obtainable by other further processing of that extract. Such a downstream product may, for example, be a topical skin treatment formulation, which may for example be in the form of a lotion, cream, gel or solution. Alternatively, the downstream product may be a food or drink product, which can for example include a capsule comprising the aqueous watercress extract of the invention or the dried UI product of the invention.

In an embodiment, the downstream product is a topical skin treatment formulation comprising the aqueous watercress extract of the invention. Optionally, the topical skin treatment formulation may further comprise a pharmaceutical carrier, for example a pharmaceutical carrier cream. Preferably, the concentration of the aqueous watercress extract in the topical skin treatment formulation would be at least 0.1 wt %, at least 0.2 wt %, at least 0.3 wt %, at least 0.4 wt %, at least 0.5 wt %, at least 0.6 wt %, at least 0.7 wt %, at least 0.8 wt %, at least 0.9 wt %, at least 1 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, or at least 25 wt %; and/or up to 100 wt %, up to 99 wt %, up to 98 wt %, up to 97 wt %, up to 96 wt %, up to 95 wt %, up to 90 wt %, up to 85 wt %, up to 80 wt %, up to 75 wt %, up to 70 wt %, up to 65 wt %, up to 60 wt %, up to 55 wt %, up to 50 wt %, up to 45 wt %, up to 40 wt %, up to 35 wt %, up to 30 wt % or up to 25 wt %.

In an embodiment, the downstream product is a topical skin treatment formulation comprising the dried UI product of the invention. Optionally, the topical skin treatment formulation may further comprise a pharmaceutical carrier, for example a pharmaceutical carrier cream. Preferably, the concentration of the dried UI product of the invention in the topical skin treatment formulation would be at least 0.001 wt %, at least 0.002 wt %, at least 0.003 wt %, at least 0.004 wt %, at least 0.005 wt %, at least 0.006 wt %, at least 0.007 wt %, at least 0.008 wt %, at least 0.009 wt %, at least 0.01 wt %, at least 0.02 wt %, at least 0.03 wt %, at least 0.04 wt %, at least 0.05 wt %, at least 0.06 wt %, at least 0.07 wt %, at least 0.08 wt %, at least 0.09 wt %, at least 0.1 wt %, at least 0.2 wt %, at least 0.3 wt %, at least 0.4 wt %, at least 0.5 wt %, at least 0.6 wt %, at least 0.7 wt %, at least 0.8 wt %, at least 0.9 wt %, at least 1 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, or at least 25 wt %; and/or up to 100 wt %, up to 99 wt %, up to 98 wt %, up to 97 wt %, up to 96 wt %, up to 95 wt %, up to 90 wt %, up to 85 wt %, up to 80 wt %, up to 75 wt %, up to 70 wt %, up to 65 wt %, up to 60 wt %, up to 55 wt %, up to 50 wt %, up to 45 wt %, up to 40 wt %, up to 35 wt %, up to 30 wt %, up to 25 wt %, up to 20 wt %, up to 15 wt %, up to 10 wt %, up to 9 wt %, up to 8 wt %, up to 7 wt %, up to 6 wt %, up to 5 wt %, up to 4 wt %, up to 3 wt %, up to 2 wt %, up to 1 wt %, up to 0.9 wt %, up to 0.8 wt %, up to 0.7 wt %, up to 0.6 wt %, up to 0.5 wt %, up to 0.4 wt %, up to 0.3 wt %, up to 0.2 wt %, or up to 0.1 wt %.

Another aspect of the invention provides the aqueous watercress extract of the invention, the fermented aqueous watercress product of the invention, the dried UI product or other product obtainable by processing of the aqueous watercress extract of the invention, for use in therapy.

Suitably, the aqueous watercress extract of the invention, the fermented aqueous watercress product of the invention, the dried UI product of the invention or other product obtainable by processing of the aqueous watercress extract of the invention may be for use in the treatment or prevention of dermatitis or skin rashes (such as e.g. nappy rash), kidney stones, urinary tract infections, hepatic encephalopathy, incontinence, sarcopenia (age-related muscle degeneration), or in the treatment of Helicobacter pylori infections.

In an embodiment, the aqueous watercress extract of the invention, the fermented aqueous watercress product of the invention, the dried UI product of the invention or other product obtainable by processing of the aqueous watercress extract of the invention is for use in the treatment or prevention of halitosis and/or chronic kidney disease.

In an embodiment, the aqueous watercress extract of the invention, the fermented aqueous watercress product of the invention, the dried UI product of the invention or other product obtainable by processing of the aqueous watercress extract of the invention is for use in cancer treatment, preferably for use in improving the sensitivity of cancer to treatment.

In an embodiment, the aqueous watercress extract of the invention, the fermented aqueous watercress product of the invention, the dried UI product of the invention or other product obtainable by processing of the aqueous watercress extract of the invention is for use as an anti-inflammatory agent.

In an embodiment, the aqueous watercress extract of the invention, the fermented aqueous watercress product of the invention, the dried UI product of the invention or other product obtainable by processing of the aqueous watercress extract of the invention is for use as an anti-microbial agent.

In an embodiment, the aqueous watercress extract of the invention, the fermented aqueous watercress product of the invention, the dried UI product of the invention or other product obtainable by processing of the aqueous watercress extract of the invention is for use as an anti-carcinogenic.

In an embodiment, the aqueous watercress extract of the invention, the fermented aqueous watercress product of the invention, the dried UI product of the invention or other product obtainable by processing of the aqueous watercress extract of the invention is for use in the treatment or prevention of antimicrobial resistance, for example by reducing antimicrobial resistance.

In an embodiment, the aqueous watercress extract of the invention, the fermented aqueous watercress product of the invention, the dried UI product of the invention or other product obtainable by processing of the aqueous watercress extract of the invention is for use in the treatment or prevention of biofilms, for example by reducing biofilms.

Suitably, the aqueous watercress extract of the invention, the fermented aqueous watercress product of the invention, the dried UI product of the invention or other product obtainable by processing of the aqueous watercress extract of the invention is for application dermally, for example in the form of a cream, lotion or gel. Suitably, the extract or the product may be applied in the form of a topical skin treatment formulation, for example a topical skin treatment formulation as described above.

A further aspect of the invention provides the use of the aqueous watercress extract of the invention, the fermented aqueous watercress product of the invention, the dried UI product of the invention or other product obtainable by processing of the aqueous watercress extract of the invention in treatment for cosmetic purposes. The aqueous watercress extract of the invention, the fermented aqueous watercress product of the invention, the dried UI product of the invention or other product obtainable by processing of the aqueous watercress extract of the invention may, for example, provide a skin soothing effect. Preferably, the extract or product may be applied dermally, for example in the form of a cream, lotion or gel. Suitably, the extract or product may be applied in the form of a topical skin treatment formulation, for example a topical skin treatment formulation as described above.

Also provided are methods of treatment corresponding to the therapeutic or cosmetic uses of the invention.

Another aspect of the invention provides the use of the aqueous watercress extract of the invention, the fermented aqueous watercress product of the invention, the dried UI product of the invention or other product obtainable by processing of the aqueous watercress extract of the invention in a food or drink product.

A further aspect of the invention provides the use of the aqueous watercress extract of the invention, the fermented aqueous watercress product of the invention, the dried UI product of the invention or other product obtainable by processing of the aqueous watercress extract of the invention in agricultural applications. Preferably, the extract or product may be used in soil treatment and/or as a fertiliser, which can for example be a urea-stabilising fertiliser or urease inhibitor fertiliser. Alternatively, the extract or product may be used in animal feed, for example feed for livestock such as e.g. chickens, pigs, sheep, and/or cows, or feed for pet animals such as e.g. cats, dogs, horses, rabbits, guinea pigs, hamsters, and/or fish.

A further aspect of the invention provides the use of the aqueous watercress extract of the invention, the fermented aqueous watercress product of the invention, the dried UI product of the invention or other product obtainable by processing of the aqueous watercress extract of the invention as a food preservative, such as for example in the preservation of meat products. Meat products can, for example, include bacon and/or processed meats such as sausages.

A further aspect of the invention provides the use of the aqueous watercress extract of the invention, the fermented aqueous watercress product of the invention, the dried UI product of the invention or other product obtainable by processing of the aqueous watercress extract of the invention in odour control, such as for example in aerosol odour control, in a deodorant and/or in a cleaning product.

Another aspect of the invention provides a watercress protein extract obtainable by the method of the invention described above. A further aspect provides products obtainable by further processing, such as fermenting or drying, of the watercress protein extract.

The watercress protein extract may comprise or consist of the coagulated protein fraction.

Suitably, the watercress protein extract comprises at least one protein which can naturally be found in watercress. Proteins which can naturally be found in watercress can, for example, comprise myrosinase; chlorophyll-binding proteins of the plant's photosystem complexes, which can include D1 protein and D2 protein; ribulose bisphosphate carboxylase (RuBisCO); and/or combinations thereof.

Another aspect of the invention provides a fermented protein product obtainable by fermenting the watercress protein extract of the invention.

A further aspect of the invention provides a dried protein product obtainable by drying, preferably freeze drying, the watercress protein extract of the invention or the fermented protein product of the invention.

The extracts and products obtainable via further processing of the extracts find a variety of uses. The invention provides compositions, such as, but not limited to food or drink products or supplements, comprising the protein extract or products obtainable by further processing of the extract.

Another aspect of the invention provides a downstream product comprising the watercress protein extract of the invention, the fermented protein product of the invention, the dried protein product of the invention or other product obtainable by further processing of the extract of the invention. Such a downstream product may, for example, be a food or drink product, which can for example include a capsule comprising the watercress protein extract of the invention or the dried protein product of the invention.

Another aspect of the invention provides the use of the watercress protein extract of the invention, the fermented protein product of the invention, the dried protein product of the invention, or other product obtainable by further processing of the extract of the invention in a food or drink product, for example in a protein supplement, which can e.g. be a sports supplement.

A further aspect of the invention provides the use of the watercress protein extract of the invention, the fermented protein product of the invention, the dried protein product of the invention or other product obtainable by further processing of the extract of the invention as a food preservative, such as for example in the preservation of meat products. Meat products can, for example, include bacon and/or processed meats such as sausages.

The following non-limiting examples are provided by way of illustration only.

### EXAMPLES

### Example 1

Watercress was blended until it was a homogeneous suspension.

This suspension was then heated on a stove until it started to form bubbles and steaming, with constant stirring. Solid could be seen separating from the liquid. The mixture was left lightly bubbling for a few minutes after which the whole mixture was poured through a muslin cloth.

The solid which was separated from the liquid is a mixture of insoluble fibre and coagulated protein, referred to as the watercress protein extract.

The remaining liquid is referred to as the aqueous watercress extract.

Quantitative amino acid analysis of samples was performed by Alta Bioscience Ltd (Redditch, UK) using an ion exchange separation of amino acids followed by post column detection with ninhydrin (European Pharmacopoeia Ph. Eur. Monograph 2.2.56 for amino acid analysis, Method 1 on page 88).

Table A shows the results of the amino acid analysis of amino acids obtained by acid hydrolysis, 24 hours at 110° C., of the watercress protein extract. Notes on analysis: Asn and Gin are completely converted to Asp and Glu during the acid hydrolysis of the protein. The values for Thr and Ser have been corrected for hydrolysis losses of 5% and 10% respectively. Trp usually suffers complete loss during acid hydrolysis and is not normally quantified. Cys is usually observed as cystine and its recovery is variable using standard hydrolysis conditions. The values for His are sometimes affected by co-eluting compounds from the sample. The reported values have been rounded off to either 2 or 3 significant figures, depending on peak size.

TABLE A
Amino acid analysis of watercress protein extract
after hydrolysis, 24 hours at 110° C.

| Amino Acid | nmol/mg | μg/mg | mg/mg | g/100 g |
|---|---|---|---|---|
| Cysteic acid¹ | — | — | — | — |
| Hydroxyproline¹ | — | — | — | — |
| Aspartic acid | 438 | 50.4 | 0.0504 | 5.04 |
| Threonine | 252 | 25.4 | 0.0254 | 2.54 |
| Serine | 253 | 22.0 | 0.0220 | 2.20 |
| Glutamic acid | 468 | 60.4 | 0.0604 | 6.04 |
| Proline | 242 | 23.5 | 0.0235 | 2.35 |
| Glycine | 373 | 21.3 | 0.0213 | 2.13 |
| Alanine | 340 | 24.2 | 0.0242 | 2.42 |
| Cystine | 19.0 | 4.23 | 0.00423 | 0.423 |
| Valine | 281 | 27.8 | 0.0278 | 2.78 |
| Methionine | 59.3 | 7.77 | 0.00777 | 0.777 |
| Isoleucine | 197 | 22.3 | 0.0223 | 2.23 |
| Leucine | 363 | 41.1 | 0.0411 | 4.11 |
| Tyrosine | 109 | 17.8 | 0.0178 | 1.78 |
| Phenylalanine | 187 | 27.6 | 0.0276 | 2.76 |
| Histidine | 100 | 13.8 | 0.0138 | 1.38 |
| Tryptophan¹ | — | — | — | — |
| Lysine | 243 | 31.1 | 0.0311 | 3.11 |
| Arginine | 181 | 28.2 | 0.0282 | 2.82 |
| Totals | 4100 | 449 | 0.449 | 44.9 |

¹Although listed, values for these compounds have not been assigned because specific analysis was not performed.

Table B shows the results of the analysis of the free amino acids in the watercress aqueous extract. These free amino acids were detected by direct amino acid analysis of the aqueous extract, without a hydrolysis step, since the protein had been removed from the aqueous extract as part of the process described above. The table shows the free amino acids in solution, expressed as Units/mg total weight. The reported values have been rounded off to either 2 or 3 significant figures, depending on peak size.

TABLE B
Amino acid analysis of free amino acids
in the aqueous watercress extract.

| Amino acid | nmol/mg | ng/mg | μg/mg | mg/mg |
|---|---|---|---|---|
| Taurine | — | — | — | — |
| Aspartic acid | 41.1 | 5470 | 5.47 | 0.00547 |
| Hydroxyproline | — | — | — | — |
| Threonine | 29.4 | 3500 | 3.50 | 0.00350 |
| Serine | 49.7 | 5230 | 5.23 | 0.00523 |
| Asparagine | 44.5 | 5880 | 5.88 | 0.00588 |
| Glutamic acid | 34.5 | 5070 | 5.07 | 0.00507 |
| Glutamine | 122 | 17800 | 17.8 | 0.0178 |
| Sarcosine | — | — | — | — |
| α-Aminoadipic | — | — | — | — |
| Proline | 2.28 | 262 | 0.262 | 0.000262 |
| Glycine | 3.97 | 298 | 0.298 | 0.000298 |
| Alanine | 11.4 | 1020 | 1.02 | 0.00102 |
| Citrulline | — | — | — | — |
| α-Aminobutyric | — | — | — | — |
| Valine | 45.6 | 5340 | 5.34 | 0.00534 |
| Cystine | 0.150 | 36.0 | 0.0360 | 0.0000360 |
| Methionine | 1.48 | 221 | 0.221 | 0.000221 |
| Cystathionine | — | — | — | — |
| Isoleucine | 22.5 | 2950 | 2.95 | 0.00295 |
| Leucine | 21.8 | 2860 | 2.86 | 0.00286 |
| Tyrosine | 9.52 | 1730 | 1.73 | 0.00173 |
| β-Alanine | 4.85 | 432 | 0.432 | 0.000432 |
| Phenylalanine | 16.4 | 2710 | 2.71 | 0.00271 |
| β-Aminobutyric | — | — | — | — |
| Homocysteine | — | — | — | — |
| γ-Aminobutyric | 41.3 | 4250 | 4.25 | 0.00425 |
| Hydroxylysine | — | — | — | — |
| Ornithine | 0.380 | 50.2 | 0.0502 | 0.0000502 |
| Lysine | 21.7 | 3170 | 3.17 | 0.00317 |
| 1-Methyl histidine | 8.75 | 1480 | 1.48 | 0.00148 |
| Histidine | 14.2 | 2210 | 2.21 | 0.00221 |
| Tryptophan | 4.85 | 990 | 0.990 | 0.000990 |
| 3-Methyl histidine | — | — | — | — |
| Anserine | — | — | — | — |
| Camosine | — | — | — | — |
| Arginine | 15.6 | 2720 | 2.72 | 0.00272 |
| Totals | 567 | 75600 | 75.6 | 0.0756 |

Tables A and B show that, in both the watercress protein extract and the aqueous watercress extract, a large range of amino acids was detected: in all cases, the contents of free amino acids in the aqueous extract (expressed relative to total weight) were lower than the corresponding contents of amino acids which were released from proteins by the acid hydrolysis of the watercress protein extract. Nevertheless, the measured contents of free amino acids in the watercress aqueous extract were substantial.

### Example 2

### Materials and Methods

### Sample Preparation

Watercress used in this trial was obtained from the Watercress Company (Dorchester, Dorset, United Kingdom). The watercress was 10 kg of dark green variety, harvested on Saturday 11 July 2020 to a specification of 20 cm length. Samples were received on Tuesday 14 July 2020. Watercress was refrigerated in a cold room at 4° C. for 24 hours before trials began.

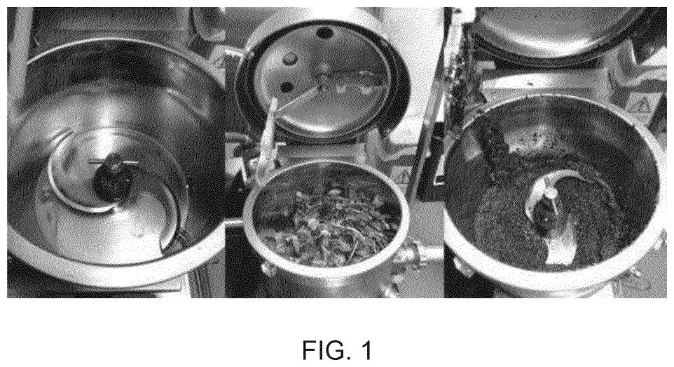

The watercress was prepared using a Roboqbo Qbo15-4 food processor, which uses a cutting blade. Cutting conditions of a speed of 2000 rpm and residence time of 5 minutes were used to homogenise the material (see FIG. 1).

Using these cutting conditions, samples of the homogenised mixture were taken at time intervals 0 (immediately after cutting), 5 min, 10 min, 30 min, 1 h, 2 h, 3 h, 6 h and 7 h. Samples were collected and sealed in a 50 mL sample tube then immersed in a water bath at 80° C. for 1 hour. Samples were then centrifuged at 14,000 g for 10 min and filtered through a 0.25 μm (PTFE) syringe filter. The filtrate was collected and 1 mL aliquots transferred to Eppendorf tubes and stored at −20° C. before analysis.

This methodology was repeated for watercress that had been stored at +4° C. for 1 week to assess the impact on its urease inhibitory (UI) activity.

### Analysis by UHPLC-QTOF Mass Spectrometry (Ultra High Performance Liquid Chromatography-Quadrupole Time-of-Flight Mass Spectrometry).

The analysis of the extracts was performed using an Agilent 1290 Infinity ultra high performance liquid chromatography (UHPLC) system equipped with an UV detector. Chromatographic separation was achieved using a Waters Acquity BEH C18 (1.7 μm×2.1 mm×100 mm) column, with a sample injection volume of 1.0 μL. A mobile phase of 1% v/v methanol/water (0.1% v/v formic acid in both solvents) was used at a flow rate of 0.25 ml/min, with the column temperature maintained at 30° C. The UHPLC was coupled to an Agilent 6530 Accurate-Mass Q-TOF LC/MS instrument. Mass spectra were collected following electrospray ionisation (ESI), in both negative and positive ion mode, of the eluent. Spray chamber conditions were: gas temperature 250° C.; drying gas flow, 8.0 L/min; nebuliser pressure, 30 psig; capillary voltage (Vcap), 4500V; nozzle voltage, 500 V; fragmentor voltage, 150 V; skimmer voltage, 65 V.

Elution was monitored by UV absorbance at 254 nm, and mass spectra recorded in the mass range m/z 60-1500. Data analysis was performed using Agilent Masshunter Software. Components were identified using extracted ion scan (EIC).

### Determination of Urease Activity Using the Berthelot Colorimetric Method

To determine the urease inhibition activity of the watercress extracts, a urease activity assay kit was purchased (MAK120 kit, Sigma Aldrich). Urease from Canavalia ensiformis (Jack Bean) was purchased from Sigma Aldrich (U1500-20KU). Analysis of the extract was performed with a microplate reader (Multiskan, ThermoFisher Scientific) using a fixed wavelength absorbance measurement at 670 nm (A₆₇₀). Urease solution (4 μg/mL) was prepared in phosphate buffer (pH 7.0, 0.01 M) that was used throughout the assay.

The protocol of the MAK120 kit was modified to measure the urease inhibitory activity of the watercress extract. 90 μL of watercress extract (prepared as set out above) was aliquoted into a 96 well plate followed by 90 μL of urease solution (for blank and standard samples, 90 μL of buffer was used instead of urease solution). 10 μL of urea was then spiked into each well and incubated at room temperature for 1 hour at 30° C. A calibration curve of ammonium chloride was prepared in the range of 0-500 M to quantitate the ammonia produced in the watercress samples.

After an hour, the reaction was terminated via the addition of 100 μL of Reagent A (sulfuric acid). The plate was briefly agitated before the addition of Reagent B. The plate was incubated at 30° C. and agitated for 30 minutes whilst protected from light. After incubation, the absorbance was measured at 670 nm (A₆₇₀) and the concentration of ammonium was calculated.

### Results and Discussion

### Analysis by UHPLC-QTOF

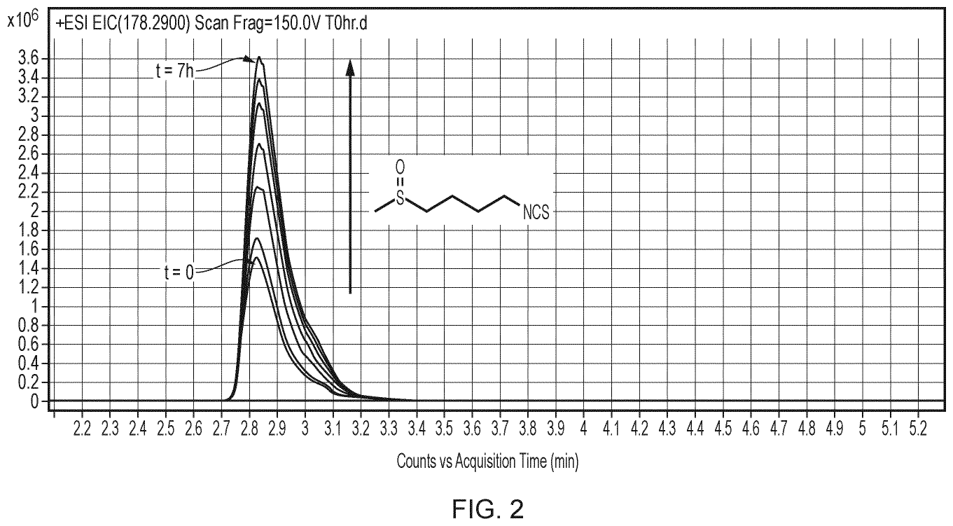

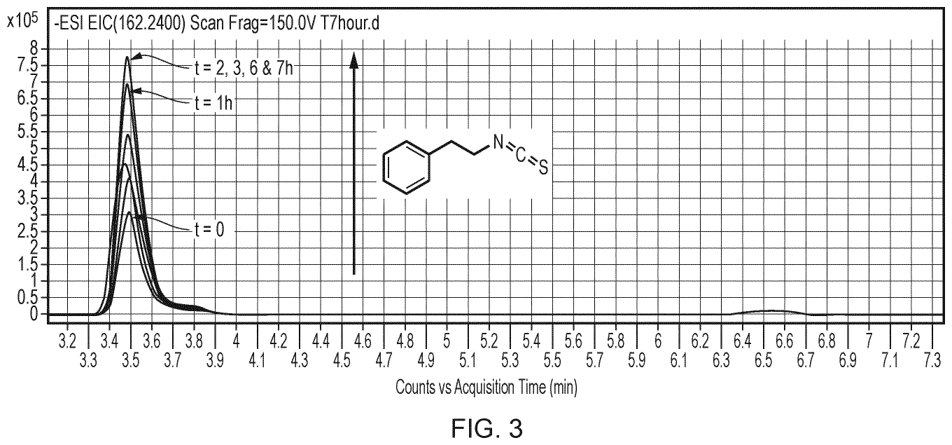

A range of components that were of interest were identified by UHPLC-QTOF mass spectrometry. These analytes were screened in positive and negative modes and tentatively identified using the extracted ion chromatogram (EIC) function. For examples of the observed EICs, see FIGS. 2 and 3.

The results of the UHPLC-QTOF mass spectrometry analysis of the composition of the watercress aqueous extract—generated in positive mode—are set out in Table 1.

The results of the UHPLC-QTOF mass spectrometry analysis of the composition of the watercress aqueous extract—generated in negative mode—are set out in Table 2.

A compilation of the UHPLC-QTOF mass spectrometry analysis of the composition of the watercress aqueous extract—generated in positive and negative mode—is set out in Table 3.

Tables 1-3 define the composition of the watercress aqueous extract, as produced by the process described above. Tables 1-3 show the results of analyses by ultra high performance liquid chromatography (UHPLC) coupled with detection by quadrupole time-of-flight (QTOF) mass spectrometry. The charged ions, as generated by electrospray ionisation (ESI), were detected as either positively charged ions (Table 1) or negatively charged ions (Table 2) by use of the instrument in positive ion mode or negative ion mode.

The peak heights of some of the detected compounds-components of the watercress aqueous extract-were found to vary with time. Examples of this are shown in FIGS. 2 and 3, in the cases of the putatively assigned peaks of the isothiocyanates, sulforaphane and phenethyl isothiocyanate (PEITC). A change in peak intensity is notated in Tables 1 to 3 with (↑) and (↓). An increase in peak intensity (notated with ↑) is relative to the peak area of the previous time point. As shown in FIG. 2, it was found that the peak intensity of the sulforaphane product ion, [M+H]⁺, at 178.29, increased over the course of 7 hours. These data suggest that the concentration of sulforaphane increases over the sampling duration. The putatively assigned component of the aqueous watercress extract mentioned above, phenethyl isothiocyanate (PEITC), is a widely-reported compound in this plant. As shown in FIG. 3, it was found that the extracted ion, [M−H]⁻ at 162.24, increases over the time period from t=0 to t=2 hours, after which the peak intensity plateaus and remains relatively constant.

TABLE 1
Analysis of watercress aqueous extract - UHPLC-QTOF data generated in positive mode

| | | Product | | Retention | | | | | |
|---|---|---|---|---|---|---|---|---|---|
| | | ion | Molecular | time | Timepoint | | | | |
| Component | Mass | [M + H]⁺ | Formula | (minutes) | 0 | 5 min | 10 min | 30 min | 1 hour |
| Glucobrassicin derivatives | | | | | | | | | |
| 4-Hydroxyglucobrassicin | 464.50 | 465.50 | C16H20N2O10S2 | 5.2 | (+) | (+) | (+) | (+) | (+) |
| Isothiocyanates (ITC's) | | | | | | | | | |
| PTCG (β-phenylethyl-glutathione) | 471.23 | 470.23 | C19H26N4O6S2 | 6.0 | (+) | (+) (↓) | (+) (↓) | (+) (↓) | (+) (↓) |
| 7-methylsulfinylheptyl | 219.37 | 220.37 | C9H17NOS2 | 7.9 | (+) | (+) (↑) | (+) (↑) | (+) (↓) | (+) (↓) |
| isothiocyanate | | | | | | | | | |
| 8-methylsulfinyloctyl | 233.40 | 234.40 | C10H19NOS2 | 8.6 | (+) | (+) (↑) | (+) (↓) | (+) (↓) | (+) (↓) |
| isothiocyanate | | | | | | | | | |
| Sulforaphane | 177.29 | 178.29 | C6H11NOS2 | 2.8 | (+) | (+) ↑ | (+) ↑ | (+) ↑ | (+) ↑ |
| Other components | | | | | | | | | |
| Tryptophan | 204.23 | 205.23 | C11H12N2O2 | 3.9 | (+) | (+) | (+) | (+) | (+) |
| Patchouli alcohol | 222.36 | 223.36 | C15H26O | 3.7 | (+) | (+) ↑ | (+) ↑ | (+) ↑ | (+) ↑ |

| | | Product | | Retention | | | | |
|---|---|---|---|---|---|---|---|---|
| | | ion | Molecular | time | Timepoint | | | |
| Component | Mass | [M + H]⁺ | Formula | (minutes) | 2 hour | 3 hour | 6 hour | 7 hour |
| Glucobrassicin derivatives | | | | | | | | |
| 4-Hydroxyglucobrassicin | 464.50 | 465.50 | C16H20N2O10S2 | 5.2 | (+) | (+) | (+) | (+) |
| Isothiocyanates (ITC's) | | | | | | | | |
| PTCG (β-phenylethyl-glutathione) | 471.23 | 470.23 | C19H26N4O6S2 | 6.0 | (+) (↓) | (+) (↓) | (+) (↓) | (+) (↓) |
| 7-methylsulfinylheptyl | 219.37 | 220.37 | C9H17NOS2 | 7.9 | (+) | (+) | (+) (↓) | (+) (↓) |
| isothiocyanate | | | | | | | | |
| 8-methylsulfinyloctyl | 233.40 | 234.40 | C10H19NOS2 | 8.6 | (+) (↓) | (+) (↓) | (+) (↓) | (+) (↓) |
| isothiocyanate | | | | | | | | |
| Sulforaphane | 177.29 | 178.29 | C6H11NOS2 | 2.8 | (+) ↑ | (+) ↑ | (+) ↑ | (+) ↑ |
| Other components | | | | | | | | |
| Tryptophan | 204.23 | 205.23 | C11H12N2O2 | 3.9 | (+) | (+) | (+) | (+) |
| Patchouli alcohol | 222.36 | 223.36 | C15H26O | 3.7 | (+) ↑ | (+) (↓) | (+) (↓) | (+) (↓) |

(+) Tentative assignment, based on chromatographic elution time and mass spectral analysis,
(↑) Concentration increasing with time,
(↓) Concentration decreasing with time

TABLE 2
Analysis of watercress aqueous extract - UHPLC-QTOF data generated in negative mode

| | | Product | | Retention | | | | | |
|---|---|---|---|---|---|---|---|---|---|
| | | ion | Molecular | time | Timepoint | | | | |
| Component | Mass | [M − H]⁻ | Formula | (minutes) | 0 | 5 min | 10 min | 30 min | 1 hour |
| Glucobrassicin derivatives | | | | | | | | | |
| 1-Methoxyglucobrassicin | 478.50 | 477.50 | C17H22N2O10S2 | 4.7 | (+) | (+) | (+) | (+) | (+) |
| 4-Methoxyglucobrassicin | 478.50 | 477.50 | C17H22N2O10S2 | 4.7 | (+) | (+) | (+) | (+) | (+) |
| 4-Hydroxyglucobrassicin | 464.50 | 463.50 | C16H20N2O10S2 | 5.2 | (+) | (+) (↑) | (+) (↑) | (+) (↑) | (+) (↑) |
| Isothiocyanates (ITC's) | | | | | | | | | |
| Phenethyl isothiocyanate (PEITC) | 163.24 | 162.24 | C9H9NS | 3.5 | (+) | (+) (↑) | (+) (↑) | (+) (↑) | (+) (↑) |
| Other components | | | | | | | | | |
| Tryptophan | 204.23 | 203.23 | C11H12N2O2 | 3.9 | (+) | (+) | (+) | (+) | (+) |

| | | Product | | Retention | | | | |
|---|---|---|---|---|---|---|---|---|
| | | ion | Molecular | time | Timepoint | | | |
| Component | Mass | [M − H]⁻ | Formula | (minutes) | 2 hour | 3 hour | 6 hour | 7 hour |
| Glucobrassicin derivatives | | | | | | | | |
| 1-Methoxyglucobrassicin | 478.50 | 477.50 | C17H22N2O10S2 | 4.7 | (+) | (+) | (+) | (+) |
| 4-Methoxyglucobrassicin | 478.50 | 477.50 | C17H22N2O10S2 | 4.7 | (+) | (+) | (+) | (+) |
| 4-Hydroxyglucobrassicin | 464.50 | 463.50 | C16H20N2O10S2 | 5.2 | (+) (↑) | (+) (↑) | (+) (↑) | (+) (↑) |
| Isothiocyanates (ITC's) | | | | | | | | |
| Phenethyl isothiocyanate (PEITC) | 163.24 | 162.24 | C9H9NS | 3.5 | (+) | (+) | (+) | (+) |
| Other components | | | | | | | | |
| Tryptophan | 204.23 | 203.23 | C11H12N2O2 | 3.9 | (+) | (+) | (+) | (+) |

(+) Tentative assignment, based on chromatographic elution time and mass spectral analysis,
(↑) Concentration increasing with time,
(↓) Concentration decreasing with time

TABLE 3
Analysis of watercress aqueous extract - compilation of
UHPLC-QTOF data generated in positive and negative mode

| | | | Retention | | | | | |
|---|---|---|---|---|---|---|---|---|
| | Molecular | | time | Timepoint | | | | |
| Component | Formula | Ionisation | (minutes) | 0 | 5 min | 10 min | 30 min | 1 hour |
| Glucobrassicin derivatives | | | | | | | | |
| 1 -Methoxyglucobrassicin | C17H22N2O10S2 | Negative | 4.7 | (+) | (+) | (+) | (+) | (+) |
| 4-Methoxyglucobrassicin | C17H22N2O10S2 | Negative | 4.7 | (+) | (+) | (+) | (+) | (+) |
| 4-Hydroxyglucobrassicin | C16H20N2O10S2 | Both | 5.2 | (+) | (+)(↑) | (+)(↑) | (+)(↑) | (+)(↑) |
| Isothiocyanates (ITC's) | | | | | | | | |
| PTCG (β-phenylethyl-glutathione) | C19H26N4O6S2 | Positive | 6.0 | (+) | (+) (↓) | (+) (↓) | (+) (↓) | (+) (↓) |
| Phenethyl isothiocyanate (PEITC) | C9H9NS | Negative | 3.5 | (+) | (+) (↑) | (+) (↑) | (+) (↑) | (+) (↑) |
| 7-methylsulfinylheptyl | C9H17NOS2 | Positive | 7.9 | (+) | (+) (↑) | (+) (↑) | (+) (↓) | (+) (↓) |
| isothiocyanate | | | | | | | | |
| 8-methylsulfinyloctyl | C10H19NOS2 | Positive | 8.6 | (+) | (+) (↑) | (+) (↓) | (+) (↓) | (+) (↓) |
| isothiocyanate | | | | | | | | |
| Sulforaphane | C6H11NOS2 | Positive | 2.8 | (+) | (+) ↑ | (+) ↑ | (+) ↑ | (+) ↑ |
| Other components | | | | | | | | |
| Tryptophan | C11H12N2O2 | Both | 3.9 | (+) | (+) | (+) | (+) | (+) |
| Patchouli alcohol | C15H26O | Positive | 3.7 | (+) | (+) ↑ | (+) ↑ | (+) ↑ | (+) ↑ |

| | | | Retention | | | | |
|---|---|---|---|---|---|---|---|
| | Molecular | | time | Timepoint | | | |
| Component | Formula | Ionisation | (minutes) | 2 hour | 3 hour | 6 hour | 7 hour |
| Glucobrassicin derivatives | | | | | | | |
| 1 -Methoxyglucobrassicin | C17H22N2O10S2 | Negative | 4.7 | (+) | (+) | (+) | (+) |
| 4-Methoxyglucobrassicin | C17H22N2O10S2 | Negative | 4.7 | (+) | (+) | (+) | (+) |
| 4-Hydroxyglucobrassicin | C16H20N2O10S2 | Both | 5.2 | (+)(↑) | (+)(↑) | (+)(↑) | (+)(↑) |
| Isothiocyanates (ITC's) | | | | | | | |
| PTCG (β-phenylethyl-glutathione) | C19H26N4O6S2 | Positive | 6.0 | (+) (↓) | (+) (↓) | (+) (↓) | (+) (↓) |
| Phenethyl isothiocyanate (PEITC) | C9H9NS | Negative | 3.5 | (+) | (+) | (+) | (+) |
| 7-methylsulfinylheptyl | C9H17NOS2 | Positive | 7.9 | (+) | (+) | (+) (↓) | (+) (↓) |
| isothiocyanate | | | | | | | |
| 8-methylsulfinyloctyl | C10H19NOS2 | Positive | 8.6 | (+) (↓) | (+) (↓) | (+) (↓) | (+) (↓) |
| isothiocyanate | | | | | | | |
| Sulforaphane | C6H11NOS2 | Positive | 2.8 | (+) ↑ | (+) ↑ | (+) ↑ | (+) ↑ |
| Other components | | | | | | | |
| Tryptophan | C11H12N2O2 | Both | 3.9 | (+) | (+) | (+) | (+) |
| Patchouli alcohol | C15H26O | Positive | 3.7 | (+) ↑ | (+) (↓) | (+) (↓) | (+) (↓) |

(+) Tentative assignment, based on chromatographic elution time and mass spectral analysis,
(↑) Concentration increasing with time,
(↓) Concentration decreasing with time

### Berthelot Reaction to Determine Ammonia Content Using Spectrophotometric Assay

As a positive control, three different enzyme solutions were prepared to determine the urease activity when phosphate buffer was used in place of watercress extract. Results from the assay indicated that all concentrations of urease enzyme were able to produce an ammonia concentration that was greater than 500 UM (highest point of calibration curve) when uninhibited from urease extract native to the watercress sample (i.e. when the watercress extract is not present)—see Table 4.

TABLE 4
Urease activity check

| | Enzyme | | Absorbance | Ammonium |
|---|---|---|---|---|
| Sample | concentration | | (670 nm) | concentration (μM) |
| Control A | 20 | mg/ml | >max. | >ULOQ |
| Control B | 200 | μg/ml | >max. | >ULOQ |
| Control C | 4 | μg/ml | >max. | >ULOQ |

>max.—Absorbance was greater than the maximum absorbance value that was within the working linear range of the spectrophotometer;
>ULOQ—Greater than upper limit of quantification (500 μM)

Two control samples were prepared to assess whether there were any interferences that might have been present in the watercress extract. Both samples contained watercress extract but did not have urease or urea added—see Table 5.

TABLE 5
Analysis of watercress aqueous extract - Watercress
extract matrix interference check.

| | | Absorbance | Ammonium |
|---|---|---|---|
| Sample | Description | (670 nm) | concentration (μM) |
| Control D | Extract + urease − urea | 0.3801 | >LLOQ |
| Control E | Extract − urease + urea | 0.1080 | >LLOQ |

<LLOQ—Less than lowest limit of quantification (0 μM)

The results in Table 5 indicate that control samples D and E had an absorbance less than that of the blank (0 μM standard). Data from the control samples A-E (Tables 4 and 5) suggest that the assay was robust and did not suffer interferences from the sample matrix.

The ammonium concentration was measured using the assay protocol outlined above under the subheading “Determination of urease activity using the Berthelot colorimetric method”. The results are shown in Table 6. This allowed the determination of the urease inhibitor (UI) activity of the aqueous watercress extract.

TABLE 6
Analysis of the aqueous watercress extract - results obtained
when the UI assay was applied to fresh watercress extracts.
The table shows the results from testing different time intervals
(t = 0 through to t = 7 h) between blending the watercress
and heating (80° C., 1 h) the resulting homogenate.

| | | | | Ammonium |
|---|---|---|---|---|
| Data | | | Absorbance | concentration |
| Set | Description | Sample | (670 nm) | (μM) |
| A | Calibration | Blank 1 | 0.2919 | N.A |
| | standards | Cal A (500 μM) | 1.3371 | |
| | | Cal B (400 μM) | 1.1766 | |
| | | Cal C (300 μM) | 0.9570 | |
| | | Cal D (200 μM) | 0.7529 | |
| | | Cal E (150 μM) | 0.6293 | |
| | | Cal F (100 μM) | 0.5186 | |
| | | Cal G (50 μM) | 0.4108 | |
| | | Cal H (0 μM) | 0.3059 | |
| B | No dilution of | t = 0 | 0.2133 | <LLOQ |
| | watercress | t = 5 min | 0.2636 | |
| | extracts | t = 10 min | 0.1937 | |
| | 4 mg/mL | t = 30 min | 0.1642 | |
| | urease | t = 1 hour | 0.1361 | |
| | added. | t = 2 hour | 0.1630 | |
| | | t = 3 hour | 0.2690 | |
| | | t = 6 hour | 0.2808 | |
| | | t = 7 hour | 0.2029 | |
| C | No dilution of | t = 0 | 0.9030 | 280.2 |
| | watercress | t = 5 min | 0.8305 | 245.9 |
| | extracts | t = 10 min | 0.8976 | 277.6 |
| | 20 mg/mL | t = 30 min | 0.8302 | 245.7 |
| | urease | t = 1 hour | 0.8547 | 257.3 |
| | added. | t = 2 hour | 0.7664 | 215.5 |
| | | t = 3 hour | 0.8018 | 232.3 |
| | | t = 6 hour | 0.7423 | 204.1 |
| | | t = 7 hour | 0.9306 | 293.2 |
| D | 10x dilution | t = 0 | 0.6004 | 137.0 |
| | of watercress | t = 5 min | 0.5992 | 136.4 |
| | extracts. | t = 10 min | 0.6145 | 143.6 |
| | 4 μg/mL | t = 30 min | 0.6427 | 157.0 |
| | urease | t = 1 hour | 0.6376 | 154.6 |
| | added. | t = 2 hour | 0.6214 | 146.9 |
| | | t = 3 hour | 0.6236 | 147.9 |
| | | t = 6 hour | 0.5898 | 131.9 |
| | | t = 7 hour | 0.6019 | 137.7 |
| E | 100x dilution | t = 0 | 0.5014 | 90.1(*) |
| | of watercress | t = 5 min | 0.7648 | 214.8 |
| | extracts. | t = 10 min | 0.5671 | 121.2 |
| | 4 μg/mL | t = 30 min | 0.5906 | 132.3 |
| | urease | t = 1 hour | 0.5905 | 132.3 |
| | added. | t = 2 hour | 0.6442 | 157.7 |
| | | t = 3 hour | 0.6398 | 155.6 |
| | | t = 6 hour | 0.6775 | 173.4 |
| | | t = 7 hour | 0.7133 | 190.4 |

(*)Insufficient sample whilst aliquoting - removed from reported average;
<LLOQ—less than lower limit of quantification (0 μM)

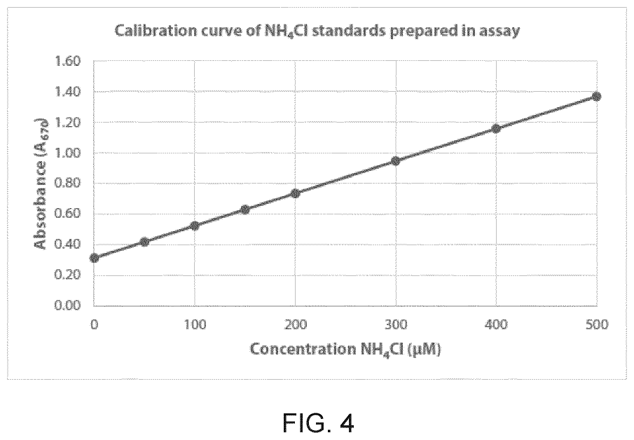

For all time points, 10- and 100-fold dilutions of the watercress extract were prepared using phosphate buffer (pH 7.0, 0.01 M) as diluent. Ammonia produced in each extract was quantified using an ammonium chloride calibration curve (see Table 6, data set A). When absorbance was plotted against concentration, a linear relationship was observed (FIG. 4).

The undiluted watercress extract, when spiked with 4 μg/mL urease enzyme and 10 UL of urea (data set B), produced an absorbance less than that of the 0 μM/phosphate blank sample. This result suggested that the urease enzyme was inhibited by the components of the watercress extract, such that the mixture was unable to convert the urea to ammonia.

When the extract was diluted 10- and 100-fold (data set D and E), it was found that the concentration of ammonium generated after 2 hours was 146.9 and 157.7 UM, respectively. The production of ammonium in diluted extract samples suggested that the urease inhibitors were not present in sufficient concentrations to fully inhibit the urease enzyme. This is summarised in Table 7.

TABLE 7
Analysis of watercress aqueous extract - UI
assay results of fresh watercress extracts

| | | Ammonium |
|---|---|---|
| | Mean average | concentration |
| | absorbance | (μM) after 2 |
| Sample | (670 nm) | hour incubation |
| Control C (phosphate buffer) | >max | >ULOQ |
| Undiluted extract | 0.21 | <LLOQ |
| 10-fold diluted extract | 0.61 | 146.9 |
| 100-fold diluted extract | 0.65 | 157.7 |

>max.—Absorbance was greater than the maximum absorbance value that was within the working linear range of the spectrophotometer;
<LLOQ—Less than lower limit of quantification (0 μM);
>ULOQ—Greater than upper limit of quantification (500 μM)

UI activity was also measured in 1-week old watercress samples that were stored at 4° C. for 1 week before processing (data not shown in table). Data collected for 1-week old watercress produced an identical result to fresh watercress extract, i.e. addition of extract fully inhibited urease, producing an absorbance that was <LLOQ (less than lower limit of quantification (0 μM)).

The application of the developed urease inhibitor (UI) assay suggests (Table 6) that, for all the tested time intervals (t=0 through to t=7 h) between blending the watercress and heating (80° C., 1 h) the resulting homogenate, there was no significant difference in their urease inhibition activity. In addition, storage of watercress at 4° C. was not shown to be detrimental to urease inhibition and produced a result comparable to that of freshly harvested watercress.

### Example 3

Watercress was blended until it was a homogeneous suspension. This suspension was then heated on a stove until it started to form bubbles and steaming, with constant stirring. Solid could be seen separating from the liquid. The mixture was left lightly bubbling for a few minutes after which the whole mixture was poured through a muslin cloth, to separate the insoluble fibre/coagulated protein from the liquid. The protein extract was freeze-dried and then analysed by Campden BRI (Chipping Campden, Gloucestershire, UK) using UKAS accredited analytical methods (unless otherwise stated; see https://www.ukas.com/wp-content/uploads/schedule_uploads/00002/1079Testing % 20Single.pdf). The analytical results are set out in Tables 8-11 below.

In the following details of the analytical methods that were used to characterise the extract, the hyphenated letters/numbers in brackets (“TES-AC . . . ”) indicate the CCFRA (Campden and Chorleywood Food Research Association) Test Number.

Protein was determined by the Kjeldahl method, using an N Factor of 6.25 (TES-AC-087). Total dietary fibre was measured by the AOAC enzymatic-gravimetric method number 991.43 (https://acnfp.food.gov.uk/sites/default/files/mnt/drupal_data/sources/files/multimedia/pdfs/annexg.pdf; TES-AC-203). Fat was determined using Weibull-Stoldt extraction (TES-AC-536). The fatty acid profile (saturates, monounsaturates (cis), polyunsaturates (cis), trans fatty acids) was determined by GC-FID (gas chromatography-flame ionisation detection; TES-AC-090). Moisture was measured by oven drying at 102° C. (TES-AC-097). Ash was determined by incineration at 525° C. (TES-AC-086). The total carbohydrate content, together with the available carbohydrate, was calculated using the nutritional data (TES-AC-335), as was the energy content. The total sugars content was measured by HPLC (high performance liquid chromatography; TES-AC-270; non-UKAS). The sugars are the sum of glucose, sucrose, maltose, lactose and fructose.

Metals and trace elements (calcium, iron, iodine (non-UKAS), zinc, magnesium, phosphorus, potassium, total sodium (and total sodium “equivalent salt”), and selenium) were determined by ICP-MS (inductively coupled plasma mass spectrometry) after pressure digestion (TES-AC-686).

B vitamins (B1, thiamine; B2, riboflavin; B3, niacin; B6, pyridoxine) were determined by LC-MS-MS (liquid chromatography with tandem mass spectrometry detection; TES-AC-713). Vitamin C and beta-carotene were measured by LC-MS (TES-AC-745 and TES-AC-021, respectively, both non-UKAS). Vitamin E (alpha-tocopherol) was measured by LC-MS-MS (TES-AC-778). Vitamin K1 and vitamin K2 were measured by LC-MS (TES-AC-751; non-UKAS). Finally, the protein extract was hydrolysed and subjected to amino acid analysis (Table 11).

TABLE 8
Analytical results for the freeze-dried fibre/coagulated protein
fraction, in relation this fraction's composition of energy,
fats, carbohydrates, fibre, protein, sodium and water. The sugars
are the sum of glucose, sucrose, maltose, lactose and fructose.

| Test | Value |
|---|---|
| Energy (kJ) | 1197 kJ/100 g |
| Energy (kcal) | 286 kcal/100 g |
| Fat (Weibull-Stoldt) | 2.4 g/100 g |
| Fatty acids saturates | 0.8 g/100 g |
| Fatty acids monounsaturates (cis) | 0.1 g/100 g |
| Fatty acids polyunsaturates (cis) | 1.4 g/100 g |
| Fatty acids trans | <0.1 g/100 g |
| Carbohydrate total | 43.7 g/100 g |
| Carbohydrate available | 9.9 g/100 g |
| Sugars (HPLC) total | 2.5 g/100 g |
| Total dietary fibre (AOAC) | 33.8 g/100 g |
| Protein (Kjeldahl) | 39.4 g/100 g |
| Protein (Kjeldahl) N Factor | 6.25 |
| Total Sodium | 330 mg/100 g |
| Total Sodium “Equivalent salt” | 0.84 g/100 g |
| Moisture at 102° C. | 5.5 g/100 g |
| Ash at 525° C. | 8.98 g/100 g |

TABLE 9
Results of the analysis of metals and trace elements in
the freeze-dried fibre/coagulated protein fraction.

| Element | mg/kg |
|---|---|
| Calcium | 17000 |
| Magnesium | 1900 |
| Phosphorus | 4100 |
| Potassium | 16000 |
| Iron | 190 |
| Selenium | 0.8 |
| Iodine | 0.55 |
| Zinc | 39 |

TABLE 10
Results of the analysis of the vitamins in the
freeze-dried fibre/coagulated protein fraction.

| Vitamin | mg/100 g |
|---|---|
| Vitamin E * | 21.9 # |
| Carotene (beta) * | 19.3 |
| Vitamin B1 as thiamine hydrochloride * | 0.55 # |
| Vitamin B2 as riboflavin * | 1.20 # |
| Vitamin B3 as niacin * | 2.04 # |
| Vitamin B6 as pyridoxine * | 0.77 # |
| Vitamin C * | 46.5 # |
| Vitamin K1 * | 10.99 # |
| Vitamin K2 * | <0.10 # |
| Vitamin B9 folate | 0.656 |

* value has been corrected for recovery;
# mean of duplicate analyses.

TABLE 11
Results of the amino acid analysis of the freeze-
dried fibre/coagulated protein fraction.

| Amino acid | g/100 g |
|---|---|
| Aspartic acid | 3.81 |
| Threonine | 1.94 |
| Serine | 1.72 |
| Glutamic acid | 4.39 |
| Proline | 1.84 |
| Glycine | 1.90 |
| Alanine | 2.17 |
| Cystine | 0.361 |
| Valine | 2.25 |
| Methionine | 0.413 |
| Isoleucine | 1.87 |
| Leucine | 3.61 |
| Tyrosine | 1.34 |
| Phenylalanine | 2.47 |
| Histidine | 1.24 |
| Lysine | 2.64 |
| Arginine | 2.14 |
| Amino acid total | 36.1 |

### Example 4

Aqueous watercress extract was produced in accordance with the sample preparation method described in Example 2, using a sample of the homogenised mixture taken at 2 hours after cutting. The aqueous watercress extract was tested to establish if it elicited an Inflammatory response In a skin model. The study was undertaken on a validated in vitro skin model by Labskin (Labskin UK, York Biotech Campus, Sand Hutton, York, UK YO41 1LZ). The negative control (non-Irritant) was Dulbecco's phosphate buffered saline (dPBS). The positive control (irritant) was 5% (w/v) SLS (sodium lauryl sulfate). It is well known that the presence of contaminating bacteria and/or bacterial cell wall components can result in a pro-inflammatory effect of the test item. Therefore, within the experimental design, before testing the extract was sterilised using four different sterilising processes resulting in extract I, extract II, extract III, and extract IV as set out in Table 12 below. All work was performed in a laboratory facility that holds current ISO9001:2015 Certification.

TABLE 12
Tested sterilising processes involving centrifugation,
autoclaving and filtration.

| Sample | Watercress extraction | |
|---|---|---|
| name | method | Sterilisation method |
| Extract | As described in | Centrifuged (10,000 g, 10 min) |
| I | Example 2 | Autoclaved (121° C., 15 min) |
| Extract | As described in | Centrifuged (10,000 g, 10 min) |
| II | Example 2 | Filtered (0.2 μm) |
| Extract | As described in | Centrifuged (10,000 g, 10 min) |
| III | Example 2 | Autoclaved (121° C., 15 min) |
| | | Centrifuged (10,000 g, 10 min) |
| Extract | As described in | Centrifuged (10,000 g, 10 min) |
| IV | Example 2 | Autoclaved (121° C., 15 min) |
| | | Filtered (0.2 μm) |

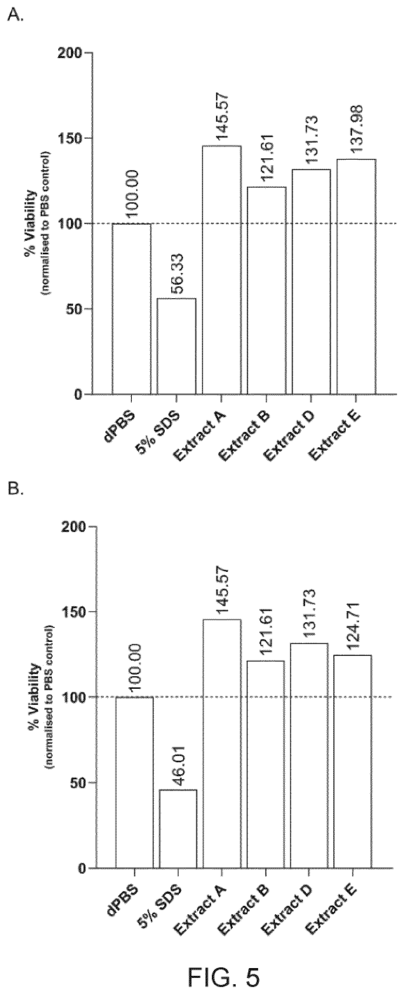

Extract I is referred to as “Extract A” in FIGS. 5, 6 and 7.
Extract II is referred to as “Extract B” in FIGS. 5, 6 and 7.
Extract III is referred to as “Extract D” in FIGS. 5, 6 and 7.
Extract IV is referred to as “Extract E” in FIGS. 5, 6 and 7.

The method to test the pro-/anti-inflammatory effects of the above extracts was as follows: primary adult human dermal fibroblasts were embedded into a fibrin matrix to produce dermal equivalents (DEs). The DEs were cultured to allow the fibroblasts to remodel the matrix. Primary neonatal human keratinocytes were applied to the DE surface and cultured under liquid for 48 h. The cells were cultured at the air liquid interface (ALI) until a stratified epidermis was formed, thereby generating the so-called “Labskin” in vitro model of human skin. Incubation conditions for all cultures was 37±2° C. in 5±1% (v/v) CO₂ at ≥95% Relative Humidity (RH).

The irritation effect of the aqueous watercress extract was assessed by following OECD test guideline 439: “In vitro skin irritation: Reconstructed Human Epidermis Test Method” (see: https://www.oecd-ilibrary.org/environment/test-no-439-in-vitro-skin-irritation-reconstructed-human-epidermis-test-method 9789264242845-en-accessed 28 Apr. 2021). Labskin units (in quintuplicates for each treatment) were treated with 11 μL of each test item (Extract I, II, III or IV) or control. Dulbecco's phosphate-buffered saline (dPBS) was the non-irritant negative control and SLS was an irritant positive control.

All Labskin units were exposed to the test items/controls for 20±1 min at 37±2° C. in 5±1% (v/v) CO₂ at ≥95% RH. After 20 min, test items were rinsed off Labskin with dPBS and the stratum corneum surface was dried with sterile filter paper. Labskin was incubated at 37±2° C. in 5±1% (v/v) CO₂ at ≥95% RH for an additional 24±2 h.

Following the 24±2 hours post-treatment incubation the assessment was performed using the following end points: (1) The undernatant was aspirated and frozen for quantification of cytokines by ELISA using the R&D systems Quantikine® ELISA for Human IL-1α/IL1F1. (2) The cytotoxicity of irritant compounds was assessed by quantification of cell viability in Labskin using an MTT (3-(4,5-dimethylthiazol-2-yl)-2,5-diphenyltetrazolium bromide) assay. To do this, the undernatant was replaced with a 1 mg·mL⁻¹ solution of MTT in Labskin maintenance medium. All Labskin units were incubated at 37±2° C. in 5±1% (v/v) CO₂ at ≥95% RH for 3.5 h. This assay is based on the ability of mitochondrial dehydrogenase enzyme from viable cells to transform the pale yellow MTT reagent to purple formazan crystals. The number of surviving cells after treatment is directly proportional to the level of formazan product created. MTT reduced to formazan was extracted from the tissue using isopropanol. The amount of formazan was quantified using a spectrophotometer at a wavelength of 540 nm. Quintuplicates of each test item were used for this purpose. The absorbance readings obtained were averaged and normalised to the negative control (dPBS) to provide the results expressed as percentage cell viability.

This test protocol allowed the prediction of the skin irritation potential of test substances according to the United Nations Globally Harmonised System (UN-GHS) for classification and labelling. A reduction of the tissue viability of equal or below 50% of the negative control, classified the substance as “category 2” (skin irritant). Tissue viability above 50% results in classification as “non-irritant”. Data handling, statistical analysis and data representation was carried out using Microsoft Excel 2016 and GraphPad Prism 8. This study was entirely carried out at Labskin facilities without the participation of any third party.

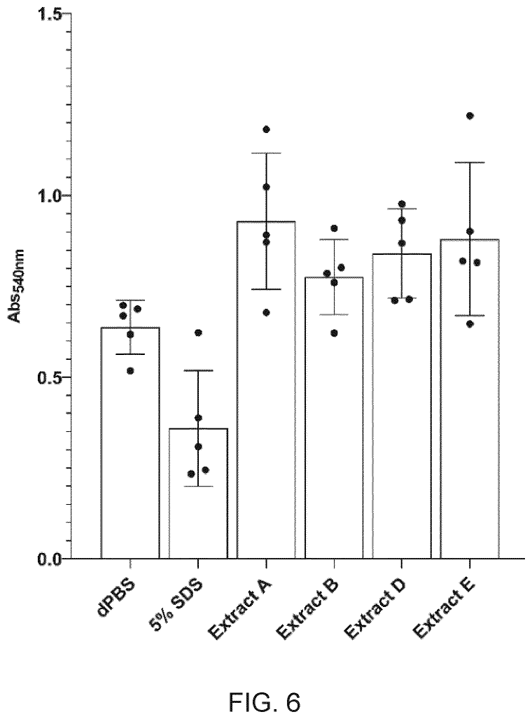

The following Results were obtained: when compared to the negative control (dPBS), Labskin exposed to a SLS solution (known irritant), reduced the viability of Labskin tissue to 56.33% (FIG. 5A). SDS falls under category 2 classification meaning the reduction should be ≥50%. The reason it was above this threshold was due to a single outlier. This outlier was removed to show a reduction of cell viability to 46.01% (FIG. 5B). There was no reduction in cell viability in the Labskin exposed to any of the test items, compared with the negative control (FIG. 5). Furthermore, there was an increase of cell viability for Labskin treated with all test items, compared to dPBS control. This result was not statistically significant. All tissue viability was statistically tested using the absorbance readings (FIG. 6 and Table 13). The reduction observed in Labskin treated with SLS was found to be statistically significant when compared to dPBS.

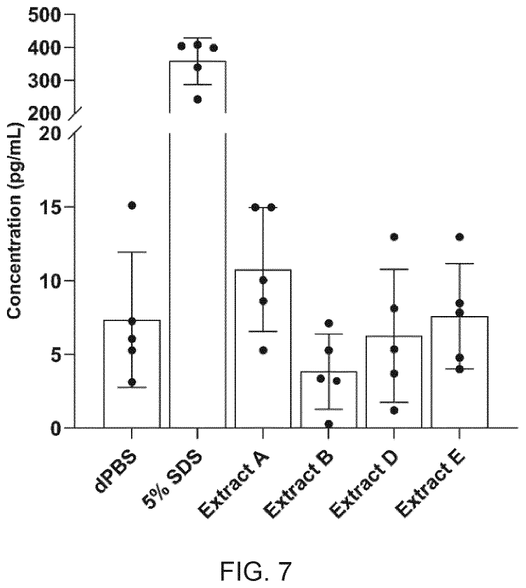

The pro-inflammatory cytokine IL-1α is known to be produced in human skin after a chemical insult occurs. To obtain additional information about the irritation effect exerted by the test items, quantification of released IL-1α was carried out using ELISA (FIG. 7). The amount of IL-1α in the undernatant correlated with the viability of the cell tissue. The Labskin exposed to SLS, released ˜48 times more IL-1α than the dPBS control. This difference was statistically significant (Table 14). In contrast, there was no significant increase in Il-1α when the Labskin was exposed to any of the extracts, compared with the dPBS control (FIG. 7 and Table 14).

TABLE 13
Statistical analysis of reduced MTT data. Tukey's multiple comparisons test of reduced MTT.

| Tukey's multiple | Mean | | | | Adjusted |
|---|---|---|---|---|---|
| comparisons test | Diff. | 95.00% CI of diff. | Significant? | Summary | P Value |
| PBS vs. 5% SDS | 0.2787 | −0.01579 to 0.5732 | No | ns | 0.071 |
| PBS vs. Extract I | −0.2908 | −0.5853 to 0.003693 | No | ns | 0.0543 |
| PBS vs. Extract II | −0.1379 | −0.4324 to 0.1566 | No | ns | 0.6987 |
| PBS vs. Extract III | −0.2025 | −0.4970 to 0.09199 | No | ns | 0.308 |
| PBS vs. Extract IV | −0.2424 | −0.5369 to 0.05209 | No | ns | 0.1507 |
| 5% SDS vs. Extract I | −0.5695 | −0.8640 to −0.2750 | Yes | **** | <0.0001 |
| 5% SDS vs. Extract II | −0.4166 | −0.7111 to −0.1221 | Yes | ** | 0.0025 |
| 5% SDS vs. Extract III | −0.4812 | −0.7757 to −0.1867 | Yes | *** | 0.0005 |
| 5% SDS vs. Extract IV | −0.5211 | −0.8156 to−0.2266 | Yes | *** | 0.0002 |
| Extract I vs. Extract II | 0.1529 | −0.1416 to 0.4474 | No | ns | 0.6031 |
| Extract I vs. Extract III | 0.0883 | −0.2062 to 0.3828 | No | ns | 0.9355 |
| Extract I vs. Extract IV | 0.0484 | −0.2461 to 0.3429 | No | ns | 0.9954 |
| Extract II vs. Extract III | −0.0646 | −0.3591 to 0.2299 | No | ns | 0.9827 |
| Extract II vs. Extract IV | −0.1045 | −0.3990 to 0.1900 | No | ns | 0.8777 |
| Extract III vs. Extract IV | −0.0399 | −0.3344 to 0.2546 | No | ns | 0.9981 |

TABLE 14
Statistical analysis of IL-1α data. Tukey's multiple comparisons test of released IL-1α.

| Tukey's multiple | Mean | 95.00% CI of | | | Adjusted |
|---|---|---|---|---|---|
| comparisons test | Diff. | diff. | Significant? | Summary | P Value |
| PBS vs. 5% SDS | −350.5 | −407.3 to −293.7 | Yes | **** | <0.0001 |
| PBS vs. Extract I | −3.408 | −60.23 to 53.42 | No | ns | >0.9999 |
| PBS vs. Extract II | 3.522 | −53.30 to 60.35 | No | ns | >0.9999 |
| PBS vs. Extract III | 1.098 | −55.73 to 57.92 | No | ns | >0.9999 |
| PBS vs. Extract IV | −0.2424 | −57.07 to 56.58 | No | ns | >0.9999 |
| 5% SDS vs. Extract I | 347.1 | 290.3 to 403.9 | Yes | **** | <0.0001 |
| 5% SDS vs. Extract II | 354 | 297.2 to 410.9 | Yes | **** | <0.0001 |
| 5% SDS vs. Extract III | 351.6 | 294.8 to 408.4 | Yes | **** | <0.0001 |
| 5% SDS vs. Extract IV | 350.3 | 293.4 to 407.1 | Yes | **** | <0.0001 |
| Extract I vs. Extract II | 6.931 | −49.89 to 63.75 | No | ns | 0.9989 |
| Extract I vs. Extract III | 4.506 | −52.32 to 61.33 | No | ns | 0.9999 |
| Extract I vs. Extract IV | 3.166 | −53.66 to 59.99 | No | ns | >0.9999 |
| Extract II vs. Extract III | −2.424 | −59.25 to 54.40 | No | ns | >0.9999 |
| Extract II vs. Extract IV | −3.765 | −60.59 to 53.06 | No | ns | >0.9999 |
| Extract III vs. Extract IV | −1.341 | −58.16 to 55.48 | No | ns | >0.9999 |

The results demonstrated that all the tested extract sterilisation methods (I, II, III, IV) did not cause a significant inflammatory response to the skin compared to the negative control. The results in FIG. 6 suggested that all four extracts increased the % viability of the cells when compared to the negative control, although this effect was not statistically significant. This may represent a beneficial skin soothing effect which is of interest in the cosmetic market. These results provided confidence that a clinical trial using the aqueous watercress extract would be safe to undertake (see Example 5).

### Example 5

With the results from Example 4 suggesting no skin irritancy effect with the aqueous watercress extract, a clinical trial was undertaken to investigate the potential of the extract in preventing inflammation when co-administered with a known skin irritant, and in treating irritation from a known skin irritant.

Aqueous watercress extract samples were produced in accordance with the sample preparation method described in Example 2, using a sample of the homogenised mixture taken at 2 hours after cutting. Before testing the samples were sterilised in accordance with the sterilisation process used for Extract II in Example 4 (centrifuged and filtered).

The samples were entered into a single-blind, within-subject clinical trial undertaken by Advanced Development & Safety Laboratories Ltd of Unit 18 Yalberton Tor Industrial Estate, Paignton, Devon, UK, TQ4 7QN (with accreditation according to ISO 9001:2015 and ISO 17025:2017), and the study was overseen by a consultant Dermatologist.

A 96-hour human patch test in 30 male and female subjects was undertaken to investigate the skin irritation potential of the extract and its effects when administered with a known skin irritant (positive control-sodium lauryl sulfate (SLS), 0.3% w/v). All subjects were healthy, consenting adults over the age of 18 of either sex. There were no exclusions or withdrawals of test subjects. The age range was 19-71 with 20 female and 10 male subjects and a Fitzpatrick Skin Assessment Range of 9-24. The study was performed in accordance with the principles of Good Clinical Research Practice and in consideration of the Helsinki Declaration (Helsinki Declaration 64th WMA General Assembly, Fortaleza, Brazil, October 2013) relating to ethical principles for research involving human subjects. It followed the “Guidelines for the Assessment of Skin Tolerance of Potentially irritant Cosmetic ingredients” (COLIPA 1997).

The study applied the following Exclusion Criteria:
Pregnant women.
Subjects with excessive blemishes, marks, scars, sunburns on the test site(s) which could interfere with scoring.
Forms of medication which may affect skin response
Signs of pre-existing skin irritation on test site(s)
Participation in simultaneous studies which that might interfere with the test evaluation
Minors

During the study the following withdrawal criteria were applied:
Subjects who did not follow the requested conditions
Subjects who suffered any illness or accident or developed any condition which could affect the outcome of the study
Subjects who no longer wished to participate in the study

Subjects were asked to refrain from exposure to UV rays and to avoid getting the patches wet and asked to provide information concerning the use of any drug, with particular reference to anti-inflammatory drugs, steroids and antihistamines. Test items (0.06 ml) were applied under occlusive dressings over a 96 h period with a wipe-off and re-apply regime every 24 h (4 applications). The patches consisted of an occlusive Finn Chamber on Scanpor 8 mm tape to which Webril (Kendall Corporation) discs, approximately 2.5 cm in diameter were fixed along the midline and applied mainly on the upper back, or upper arms when upper back was not suitable. The area of skin designated for patch testing was cleaned with demineralised water and towel dried. The patch was applied to the cleaned area.

At the applicable time-intervals the patch was removed and residue was wiped away. Approximately 15 min after removal of the patch the application area was carefully examined by the competent study monitor to evaluate skin reactions and grade accordingly with values ranging from 0 to 3 to express differences in observable reactions.

The sites were assessed by the same qualified assessor (consultant dermatologist) on all days according to the European Society of Contact Dermatitis guidelines for diagnostic patch testing (Johansen J D et al. European Society of Contact Dermatitis guideline for diagnostic patch testing-recommendations on best practice. Contact Dermatitis. 2015 October; 73 (4): 195-221. doi: 10.1111/cod.12432. Epub 2015 Jul. 14. PMID: 26179009. https://onlinelibrary.wiley.com/doi/epdf/10.1111/cod.12432). Illumination of the sites for assessment was by a 60 W pearl bulb, approximately 30 cm away from the site. Skin reactions were scored at the first, second, third and fourth dressing removals, according to the Evaluation Scale (which includes erythema, oedema, dryness/desquamation and vesicles (Table 15). Mean Daily Irritation Scores (MDIS) were calculated by adding up the evaluation grades recorded for the subjects within the panel, then dividing the total by the number of subjects. A mean value was therefore used.

TABLE 15
Scoring system for the human skin inflammatory response.
ERYTHEMA
Superficial reddening of the skin as a result of
irritation causing dilatation of the blood capillaries

| | |
|---|---|
| None | 0 |
| Light erythema, hardly noticeable | 1 |
| Moderate and uniform redness | 2 |
| Severe and uniform redness | 3 |

OEDEMA
A build-up of fluid in the body which causes
the affected tissues to become swollen

| | |
|---|---|
| None | 0 |
| Slight (edges of area well defined by definite raising) | 1 |
| Moderate (raised approximately 1 mm) | 2 |
| Severe (raised more than 1 mm/extending beyond | 3 |
| the area of exposure | |

DRYNESS/DESQUAMATION
Skin peeling/shedding of the outermost
membrane or layer of skin

| | |
|---|---|
| None | 0 |
| Dryness with light desquamation (smooth skin) | 1 |
| Moderate desquamation | 2 |
| Severe desquamation | 3 |

VESICLES
Small, fluid-filled sacs that can appear on the skin

| | |
|---|---|
| None | 0 |
| Very small vesicles (barely visible) | 1 |
| Clearly visible, small vesicles (well-defined contours) | 2 |
| Large, well-defined vesicles | 3 |

Subject data were held according to the requirement of General Data Protection Regulation (GDPR) with the data being archived electronically by Advanced Development & Safety Laboratories Limited. A residual sample of the Investigational Product (IP) was kept for c. 1 month. The information concerning the subjects required as part of undertaking the testing was confidentially treated and any photos were taken in such a manner as to deem the subject non-recognisable. Identifiable personal information of subjects was not communicated to third parties. As part of reporting the results from Patch Tests and User Evaluations, the data were anonymised.

The individual test items applied to the dermal patch were:
Neat Extract
10% Extract (diluted with deionised water)
Carrier Cream
Carrier Cream with 10% Neat Extract

$$SLS 0.3 \% + Neat Extract ( 50 : 50 )$$

$$SLS 0.3 \% + 10 \% Extract ( 50 : 50 )$$

$$SLS 0.3 \% + Carrier Cream ( 50 : 50 )$$

$$SLS 0.3 \% + Carrier Cream with 10 \% extract ( 50 : 50 )$$

48 hours positive control (SLS 0.3%) followed by 48 hours Neat Extract
48 hours positive control (SLS 0.3%) followed by 48 hours 10% Neat Extract
48 hours positive control (SLS 0.3%) followed by 48 hours Carrier Cream
48 hours positive control (SLS 0.3%) followed by 48 hours Carrier Cream with 10% Extract (50:50)
Negative control (deionised water)
Positive control (SLS 0.3%)

The following results were obtained (see Tables 16 and 17): both the neat extract and the 10% extract had a mean Daily Irritation Score (MDIS) of 0.00 at 24, 48, 72 and 96 h, with no evidence of any inflammatory response in any test subjects across the duration of the trial. The cumulative irritation potential was therefore below 0.3 which classified the extract as “not irritating”.

### Tables 16 and 17.

### Mean Daily Irritation Score (MDIS) of 21085: Neat Extract

MDIS is calculated by adding up the evaluation grades recorded for the subjects within the Panel then dividing the total by the number of subjects. A mean value is returned.

| Time | Valid | Missing | MDIS | Median | STDEV | Min | Max |
|---|---|---|---|---|---|---|---|
| IP-24 | 30 | 0 | 0.00 | 0.00 | 0.00 | 0.00 | 0.00 |
| IP-48 | 30 | 0 | 0.00 | 0.00 | 0.00 | 0.00 | 0.00 |
| IP-72 | 30 | 0 | 0.00 | 0.00 | 0.00 | 0.00 | 0.00 |
| IP-96 | 30 | 0 | 0.00 | 0.00 | 0.00 | 0.00 | 0.00 |

### Mean Daily Irritation Score (MDIS) of 21082: 10% Extract

MDIS is calculated by adding up the evaluation grades recorded for the subjects within the Panel then dividing the total by the number of subjects. A mean value is returned.

| Time | Valid | Missing | MDIS | Median | STDEV | Min | Max |
|---|---|---|---|---|---|---|---|
| IP-24 | 30 | 0 | 0.00 | 0.00 | 0.00 | 0.00 | 0.00 |
| IP-48 | 30 | 0 | 0.00 | 0.00 | 0.00 | 0.00 | 0.00 |
| IP-72 | 30 | 0 | 0.00 | 0.00 | 0.00 | 0.00 | 0.00 |
| IP-96 | 30 | 0 | 0.00 | 0.00 | 0.00 | 0.00 | 0.00 |

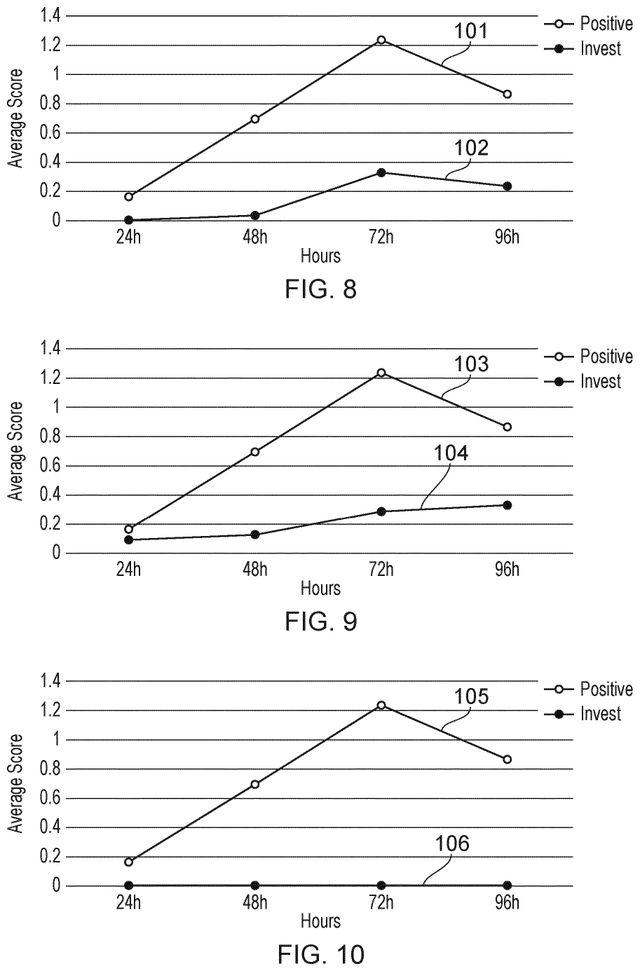

Co-administration (1:1 v/v) of the aqueous watercress extract with the positive control (0.3% SLS) significantly reduced skin irritation compared to a 1:1 dilution (using distilled water) of the positive control (p<0.05; FIG. 8).

Co-administration (1:1 v/v) of the 10% aqueous watercress extract with the positive control (0.3% SLS) significantly reduced skin irritation compared to the group which was administered the 1:1 dilution of the positive control alone (p<0.05; FIG. 9).

Co-administration (1:1 v/v) of the 10% extract (which had been formulated within a carrier cream) with the positive control (0.3% SLS) significantly reduced skin irritation compared to the group which was administered the 1:1 dilution of the positive control alone (p<0.05; FIG. 10).

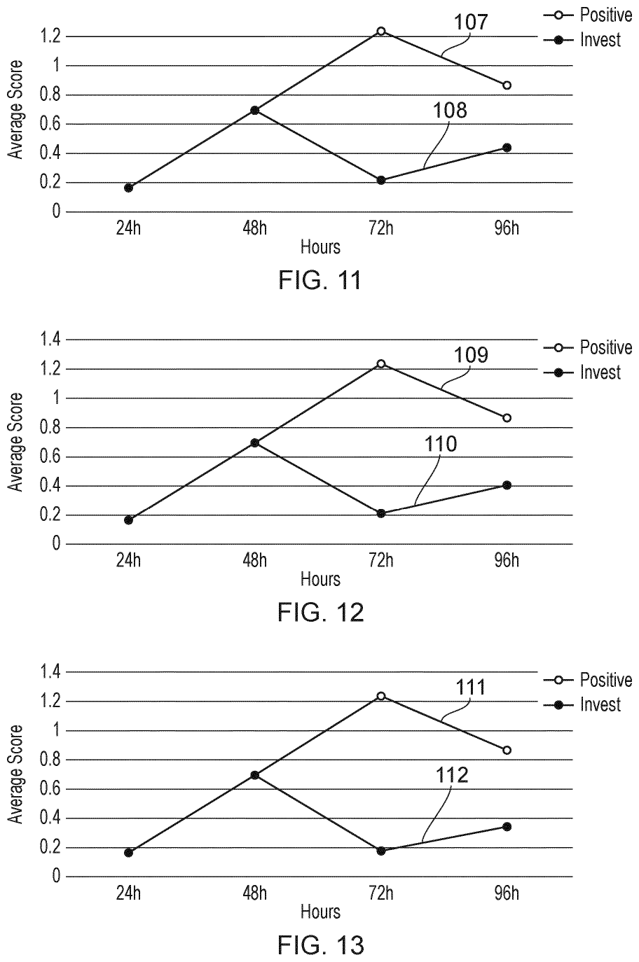

The neat extract (diluted 1:1, v/v, with distilled water; and applied 48 h after an application of the positive control) reduced skin irritation at the 72 and 96 h time-points, compared with the group in which the positive control was applied, left for 48 h, and then treated with distilled water (FIG. 11).

The 10% extract (diluted 1:1, v/v, with distilled water; and applied 48 h after an application of the positive control) reduced skin irritation at the 72 and 96 h time-points, compared with the group in which the positive control was applied, left for 48 h, and then treated with distilled water (FIG. 12).

The extract which had been formulated within a carrier cream to provide a 10%, v/v, preparation (then diluted 1:1, v/v; and applied 48 h after an application of the positive control) reduced skin irritation at the 72 and 96 h time-points, compared with the group in which the positive control was applied, left for 48 h, and then treated with distilled water (FIG. 13).

Following the trial, the following assertions are substantiated for the aqueous watercress extract: Dermatologically tested; Dermatologically approved; Clinically Tested; Safe for Skin; Suitable for sensitive skin; Suitable for all skin types.

The results demonstrate that the aqueous watercress extract, as both a neat extract and as a 10% dilution, can reduce inflammation when co-administered topically with a skin irritant. In established inflammation it may have a role in augmenting the healing process. This could be particularly beneficial in continence care and other forms of irritant dermatitis.

An observation is the reduction in inflammation seen using the Carrier Cream with 10% extract co-administered with the Positive Control when compared to the positive control alone is greater than that seen with the Neat Extract, 10% Extract or Carrier Cream separately. This suggests a beneficial additive effect of combining the carrier cream with the 10% extract. The mixture of carrier cream with 10% extract eliminated any inflammation seen with the positive control which suggests this preparation could be effective against even more potent skin irritants, and the upper limit of its function has not been observed here.

The tested aqueous watercress extracts (neat and 10%) therefore exhibit skin anti-inflammatory functions in terms of prevention and treatment. The extracts can be used on their own or combined with an existing topical carrier product. Without wishing to be bound by theory, this effect may be due, at least in part, to the PEITC created during the production process as described in Example 2, although it is likely that other constituents contribute to the anti-inflammatory effect of the aqueous watercress extract.

### Example 6

Aqueous watercress extract was produced in accordance with the sample preparation method described in Example 2. As described above, the heating step led to a visible coagulation, by which the homogenous suspension separated into two components: (1) a solid, putty-like, component and (2) a transparent brown liquid (referred to as the aqueous watercress extract). The aqueous watercress extract was tested to establish if it had the capacity to scavenge ammonia directly. An aqueous solution of ammonium chloride (NH₄Cl; 7 mM) was prepared in 100 mM sodium phosphate buffer (pH 7.4). The negative control sample contained buffer alone (no NH₄Cl added), and the positive control sample contained NH₄Cl (no watercress extract added). Aqueous watercress extract (100 μL) at desired dilutions was mixed with 7 mM NH₄Cl (100 μL) and incubated for 30 min at 37° C. Following the incubation, the amount of ammonia was determined using the Berthelot assay, as follows: 20 μL of the NH₄Cl and watercress extract mix was added to each well (including 2× technical repeats) of a 96-well microplate (Corning), where each well already contained 10 μL of 0.5% sulfuric acid. Solution A (50 μL; 106 mM phenol, 191 μM sodium nitroprusside) and solution B (50 μL; 125 mM sodium hydroxide, 125 mM sodium hypochlorite) were added to each well and the plate was incubated for 30 min at 37° C. The absorbance values (at 636 nm) of the wells in the plate were measured using a microplate reader. The percentage (%) ammonia was determined using the equation: (sample value−negative control value)/(positive control value−negative control value)×100%.

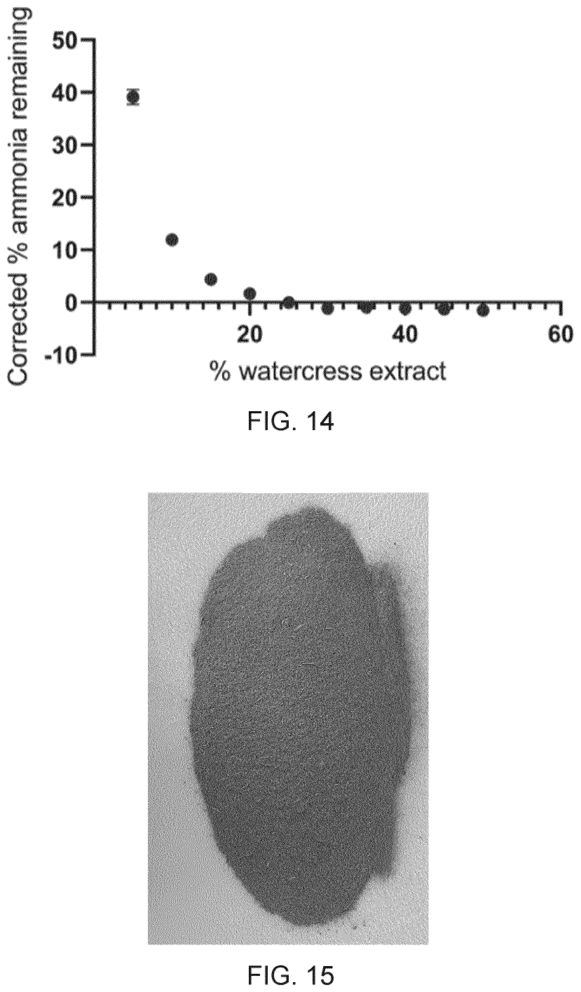

The results, which are shown in FIG. 14, demonstrate that the aqueous watercress extract has the ability to scavenge ammonia in a concentration-dependent manner. At a dilution corresponding to 10% of the neat watercress extract, about 90% of the ammonia was removed. Without wishing to be bound by theory, this direct ammonia-scavenging effect of the watercress extract may supplement the ability of the watercress extract to inhibit the urease-catalysed production of ammonia. Without wishing to be bound by theory, both—or either of—these mechanisms of action of the watercress extract may serve to lower ammonia concentrations in target systems.

### Example 7

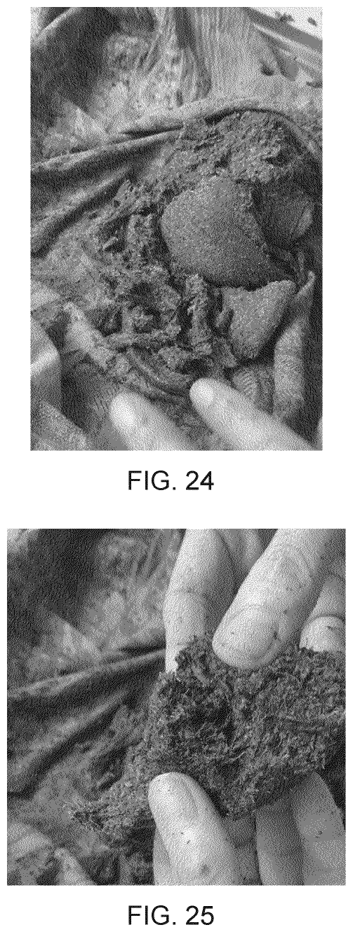

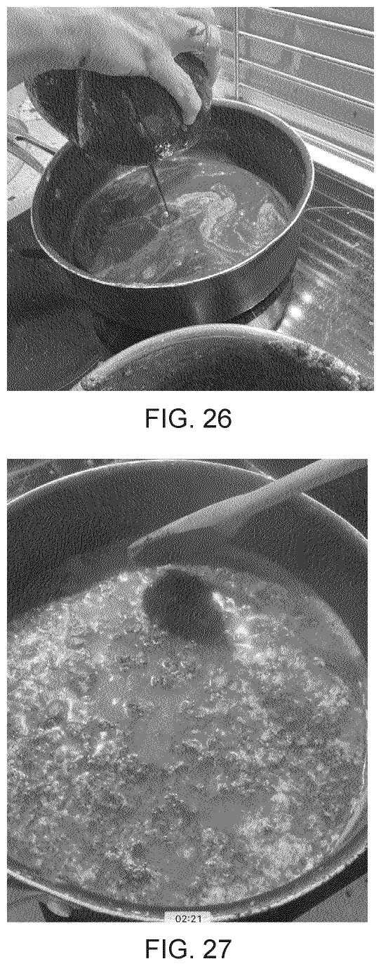

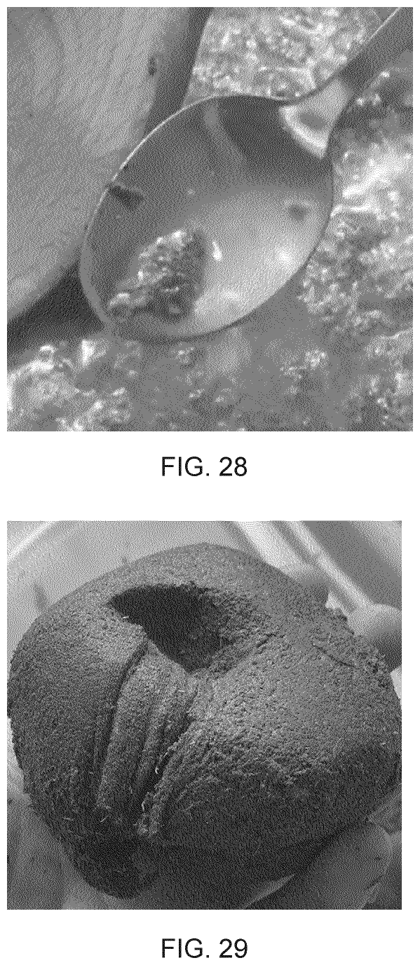

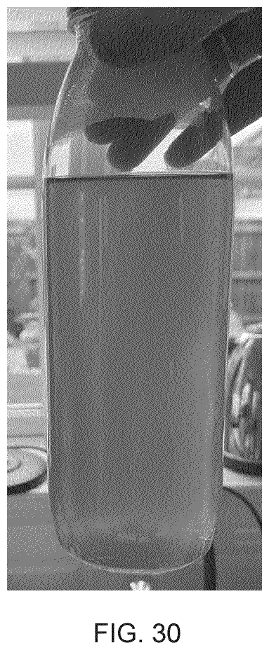

In an experiment to clearly demonstrate the coagulation effect, 500 g fresh watercress plants (leaf and stem) were coarsely chopped with a knife by hand and then blended in a kitchen blender (Kenwood FP 195 food processor) for approximately 3 minutes until there was a thick green homogeneous mixture (see FIG. 23). To improve the observable coagulation effect, the mixture was then passed through a muslin cloth to remove insoluble fibre (FIGS. 24 and 25), leaving a more bright green and less viscous liquid to collect in a pan (FIG. 26). At this point the liquid has no observable solid component. This liquid was then heated in a pan to approximately 80° C. until it began to steam. At this point an observable separation occurs whereby the protein rapidly coagulates and the homogenous green liquid separates in the pan to form a green putty-like solid and a transparent brown liquid. This is shown in the pan in FIG. 27, and close up on a spoon in FIG. 28. To further demonstrate this separation, the contents of the pan were poured through an identical muslin cloth to the earlier fibre removal stage. The solid putty-like coagulant is then left in the cloth; this is shown separately as a solid ball in FIG. 29. The brown transparent liquid which passes through the cloth is collected and bottled; this is demonstrated in FIG. 30. This process demonstrates a clear coagulation effect whereby, on heating a fibre-removed blended aqueous watercress intermediate, the soluble protein coagulates to yield a green solid putty and a separate transparent brown liquid, which are both very dissimilar to the starting liquid. This is also achievable without prior removal of the fibre but for the purposes of the images fibre removal leads to a more clearly observable coagulation effect during the process.

### Example 8

The aqueous watercress extract and the watercress protein extract were produced in accordance with the sample preparation method described in Example 1. The two extract types were freeze-dried (FreeZone 6 Freeze Dry System, Labconco, USA) and stored at −80° C. until analysis. The samples were assayed for nitrate (NO₃⁻) and nitrite (NO₂⁻) by reconstituting the dried samples with the same volume of water that had been removed by freeze-drying. For some sample analyses, a further known dilution of the sample was performed, in order provide analyte concentrations which fell within the standard curve ranges of the nitrate and nitrite assays. The reconstituted samples were mixed vigorously for 1 min and then allowed to stand in upright test tubes for 1 hour, resulting in an insoluble pellet at the base of each tube and a clear supernatant. NO₃⁻ and NO₂⁻ concentrations in the supernatants were determined using an “API Freshwater Master Test Kit” (Mars Fishcare Europe, Waltham-on-the-Wolds, UK), according to the manufacturer's instructions.

TABLE 18
Results from the analysis of nitrate and nitrite anion concentrations
in the aqueous watercress extract and the watercress protein extract.

| | Nitrate | Nitrite |
|---|---|---|
| | concen- | concen- |
| Sample name | tration (mM) | tration (mM) |
| Batch A of aqueous watercress extract | 11.7 | — |
| (not frozen or freeze-dried) | | |
| Batch B of aqueous watercress extract | 5.2 | N.D. |
| (not frozen or freeze-dried) | | |
| Aqueous watercress extract which was | 4.8 | N.D. |
| freeze-dried, then reconstituted with | | |
| the volume of water which was removed | | |
| by freeze-drying | | |
| Watercress protein extract which was | 14.4 | N.D. |
| freeze-dried, then reconstituted with | | |
| the volume of water which was removed | | |
| by freeze-drying | | |

N.D. = not detectable.

Table 18 shows the concentrations of nitrate and nitrite in different batches of aqueous watercress extract and in the watercress protein extract. There are substantial contents of nitrate in both the different types of extract. The nitrite concentration was undetectable. Without wishing to be bound by theory, these results suggest that the watercress extracts (and their corresponding fermented and/or dried products) may be useful in food preservation, especially in relation to meat products.

### Comparative Example 1

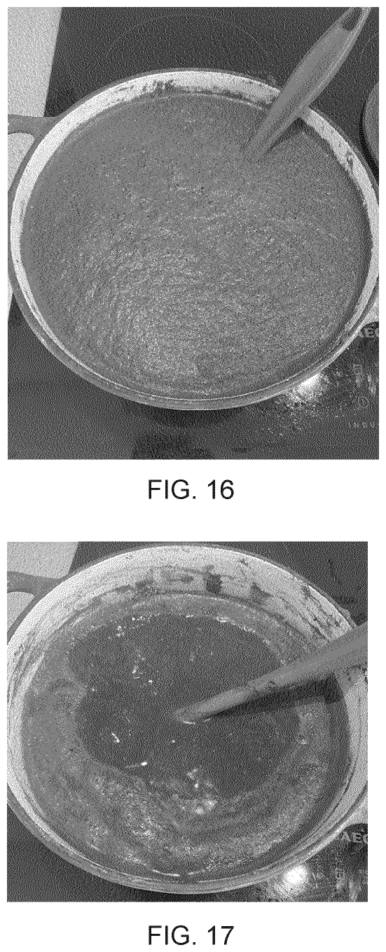

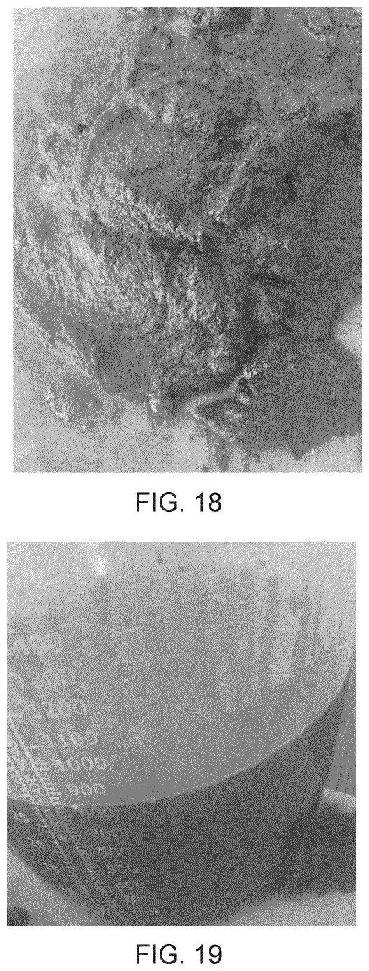

In a comparative example, a watercress extract was produced following the method disclosed in the English (Espacenet) abstract for patent document KR20000009589A. 100 g of oven dried pulverised watercress powder was bought from The Watercress Company in Dorchester, England (see FIG. 15). This was mixed with 3 litres of water and heated at 80° C. for 3 hours using a kitchen thermometer to regulate the temperature. Initially the mixture was a light green colour, and after 2 hours this had darkened (see FIGS. 16 and 17). The mixture was then filtered through a clean paper coffee filter. FIG. 18 shows the disintegrated solid matter in the filter and FIG. 19 shows the dark brown liquid. There was no observable coagulation or separation of solid and liquids throughout the liquid step.

### Comparative Example 2

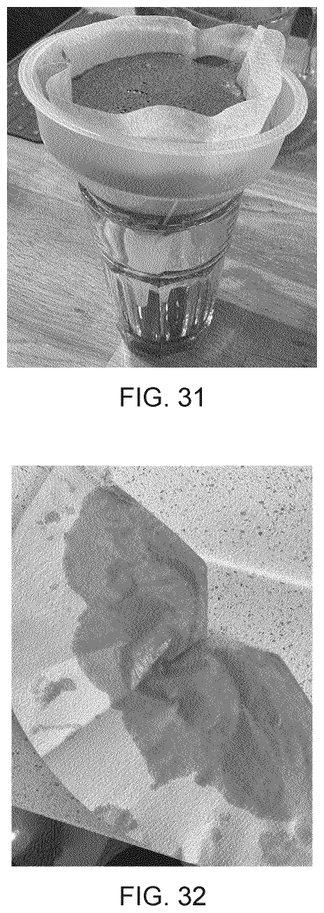

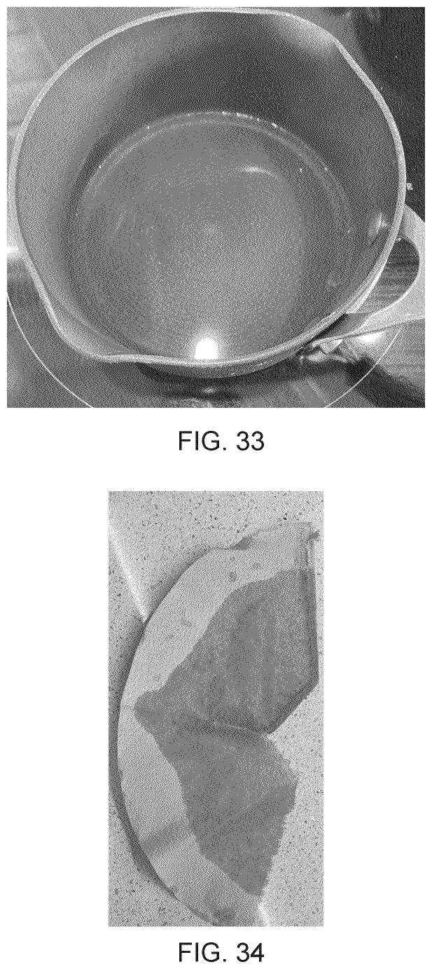

A further comparative experiment was undertaken to demonstrate that when starting with a dried watercress powder, a coagulation effect does not occur on heating. To demonstrate this, 300 g of oven dried watercress powder, bought from The Watercress Company in Dorset, England (www.thewatercresscompany.com), was mixed thoroughly with 300 ml of water using a Kenwood FP 195 food processor for 10 minutes to create a thick green mixture. The mixture was then poured into a paper coffee filter embedded within a funnel to collect a brown transparent liquid (see FIG. 31). This liquid was then poured through 2 further clean paper coffee filters, in order to remove any solids before the heating step. The third filter, shown in FIG. 32, had no visible solid particulate matter on it. The obtained liquid was then heated to 80° C. in a pan (see FIG. 33). There was no visible separation of a solid component from the liquid component. The liquid was then passed through another clean coffee filter and there was no evidence of a solid coagulant retained in the filter (see FIG. 34). This demonstrates that the dried watercress powder does not contain soluble protein and therefore a coagulation effect does not occur on heating this mixture with water. Without wishing to be bound by theory, it is thought that when starting with a dried watercress powder, the protein may already be in a denatured state and therefore is no longer water soluble. The coagulation effect may also be dependent on mobilising the protein through macerating the watercress and subsequently heating.

### Comparative Example 3

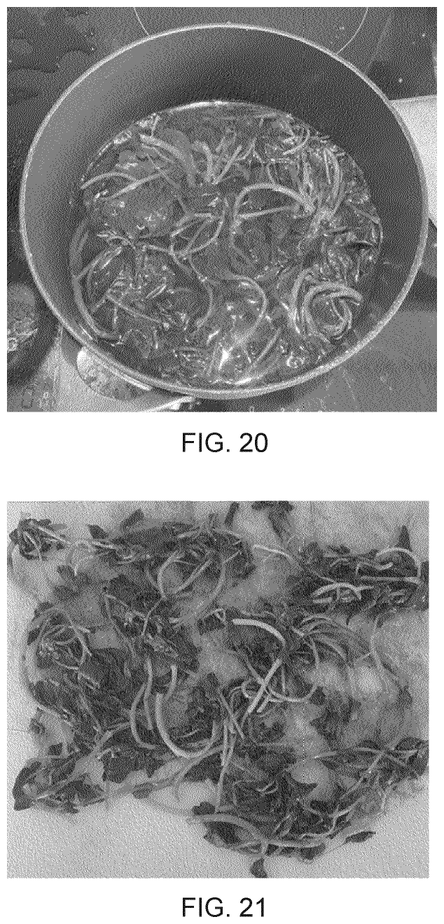

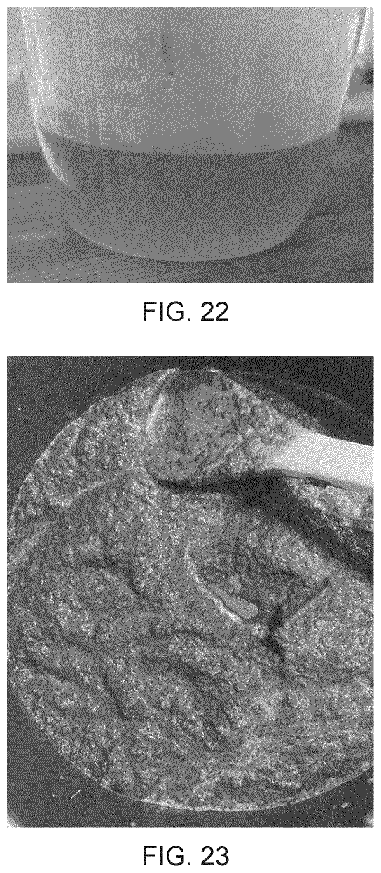

In another comparative example, watercress extract was prepared following the method disclosed in patent document GB2205038A. 3 and ⅝ ounces of fresh watercress plant leaves and stems were added to one pint of water in a pan and heated to boiling point (see FIG. 20). This was allowed to simmer for 5 minutes and was then cooled. The contents were strained through a fine muslin cloth to recover the stems and leaves (FIG. 21) and the liquid (FIG. 22). In this example there was no observable coagulation or separation of a new solid component from the liquid component upon heating.

1. A method for preparing at least one watercress extract, which method comprises:
(a) macerating fresh watercress to form an aqueous watercress intermediate;
(b) heating at least part of the aqueous watercress intermediate to coagulate at least a portion of the proteins in the fresh watercress, forming a coagulated protein fraction and a liquid fraction; and
(c) isolating the liquid fraction to provide an aqueous watercress extract and/or isolating the coagulated protein fraction to provide a watercress protein extract.

2-47. (canceled)

48. The method of claim 1, wherein macerating the fresh watercress comprises macerating a watercress component, the component comprising the fresh watercress and optionally a liquid maceration medium.

49. The method of claim 48, wherein the watercress component is substantially free of denatured watercress protein.

50. The method of claim 1, wherein in step (a), macerating the fresh watercress comprises blending the fresh watercress.

51. The method of claim 1, wherein in step (a), macerating the fresh watercress comprises contacting the fresh watercress with a blade.

52. The method of claim 1, wherein the aqueous watercress intermediate contains water which was present in the fresh watercress, as well as other components which were present in the fresh watercress.

53. The method of claim 1, wherein the method comprises removing solids from the aqueous watercress intermediate before the heating step (b).

54. The method of claim 1, wherein the heating step (b) is conducted at a temperature in the range from 50° C. to 200° C.

55. The method of claim 1, wherein the heating step (b) is conducted for a time period in the range from 2 seconds to 2 hours.

56. The method of claim 1, wherein in step (c), isolating the liquid fraction to provide an aqueous watercress extract and/or isolating the coagulated protein fraction to provide a watercress protein extract is conducted by filtration or centrifugation.

57. The method of claim 56, wherein isolating the liquid fraction to provide an aqueous watercress extract and/or isolating the coagulated protein fraction to provide a watercress protein extract is conducted by filtration, which filtration is conducted using a filter with a pore size in the range from 0.1 μm to 0.45 μm.

58. The method of claim 1, wherein in step (c), the liquid fraction is isolated to provide the aqueous watercress extract.

59. The method of claim 58, wherein the aqueous watercress extract comprises at least one urease inhibitor and/or has urease inhibitory activity.

60. The method of claim 1, wherein in step (c), the coagulated protein fraction is isolated to provide the watercress protein extract.

61. The method of claim 1, further comprising the step of sterilising the aqueous watercress extract and/or the watercress protein extract.

62. The method of claim 1, further comprising the step of fermenting the aqueous watercress extract and/or the watercress protein extract.

63. The method of claim 1, further comprising the step of drying, optionally freeze drying, the aqueous watercress extract and/or the watercress protein extract.

64. The method of claim 1, further comprising the step of blanching the watercress prior to the maceration step (a).

65. An aqueous watercress extract obtainable by the method of claim 1.

66. A watercress protein extract obtainable by the method of claim 1.
